SEQUENCE LISTING (180 sequences)

SEQ ID NO: 1 - protein, 10 aa, synthetic construct
  REGION 1..10: note = Synthetic polypeptide
GFTFSDSWIH

SEQ ID NO: 2 - protein, 18 aa, synthetic construct
  REGION 1..18: note = Synthetic polypeptide
AWISPYGGSTYYADSVKG

SEQ ID NO: 3 - protein, 9 aa, synthetic construct
  REGION 1..9: note = Synthetic polypeptide
RHWPGGFDY

SEQ ID NO: 4 - protein, 118 aa, synthetic construct
  REGION 1..118: note = Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 5 - protein, 11 aa, synthetic construct
  REGION 1..11: note = Synthetic polypeptide
RASQDVSTAVA

SEQ ID NO: 6 - protein, 7 aa, synthetic construct
  REGION 1..7: note = Synthetic polypeptide
SASFLYS

SEQ ID NO: 7 - protein, 9 aa, synthetic construct
  REGION 1..9: note = Synthetic polypeptide
QQYLYHPAT

SEQ ID NO: 8 - protein, 107 aa, synthetic construct
  REGION 1..107: note = Synthetic polypeptide
DIQMTQSPSSLSASVGDRVTITCRASQDVSTAVAWQQKPGKAPKLLIYSASFLYSGVPSRFSGSGSGTDFTLTISSLQPEDFATYYCQQYLYHPATFGQGTKVEIKR

SEQ ID NO: 9 - protein, 5 aa, synthetic construct
  REGION 1..5: note = Synthetic polypeptide
SYIMM

SEQ ID NO: 10 - protein, 17 aa, synthetic construct
  REGION 1..17: note = Synthetic polypeptide
SIYPSGGITFYADTVKG

SEQ ID NO: 11 - protein, 11 aa, synthetic construct
  REGION 1..11: note = Synthetic polypeptide
IKLGTVTTVDY

SEQ ID NO: 12 - protein, 120 aa, synthetic construct
  REGION 1..120: note = Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 13 - protein, 14 aa, synthetic construct
  REGION 1..14: note = Synthetic polypeptide
TGTSSDVGGYNYVS

SEQ ID NO: 14 - protein, 7 aa, synthetic construct
  REGION 1..7: note = Synthetic polypeptide
DVSNRPS

SEQ ID NO: 15 - protein, 10 aa, synthetic construct
  REGION 1..10: note = Synthetic polypeptide
SSYTSSSTRV

SEQ ID NO: 16 - protein, 110 aa, synthetic construct
  REGION 1..110: note = Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 17 - protein, 10 aa, synthetic construct
  REGION 1..10: note = Synthetic polypeptide
GFTFSRYWMS

SEQ ID NO: 18 - protein, 17 aa, synthetic construct
  REGION 1..17: note = Synthetic polypeptide
NIKQDGSEKYYVDSVKG

SEQ ID NO: 19 - protein, 12 aa, synthetic construct
  REGION 1..12: note = Synthetic polypeptide
EGGWFGELAFDY

SEQ ID NO: 20 - protein, 121 aa, synthetic construct
  REGION 1..121: note = Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 21 - protein, 12 aa, synthetic construct
  REGION 1..12: note = Synthetic polypeptide
RASQRVSSSYLA

SEQ ID NO: 22 - protein, 7 aa, synthetic construct
  REGION 1..7: note = Synthetic polypeptide
DASSRAT

SEQ ID NO: 23 - protein, 9 aa, synthetic construct
  REGION 1..9: note = Synthetic polypeptide
QQYGSLPWT

SEQ ID NO: 24 - protein, 108 aa, synthetic construct
  REGION 1..108: note = Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAWYYCQQYGSLPWTFGQGTKVEIK

SEQ ID NO: 25 - protein, 13 aa, synthetic construct
  REGION 1..13: note = Synthetic polypeptide
KASGGTFSSYAIS

SEQ ID NO: 26 - protein, 17 aa, synthetic construct
  REGION 1..17: note = Synthetic polypeptide
GIIPIFGTANYAQKFQG

SEQ ID NO: 27 - protein, 16 aa, synthetic construct
  REGION 1..16: note = Synthetic polypeptide
ARSPDYSPYYYYGMDV

SEQ ID NO: 28 - protein, 123 aa, synthetic construct
  REGION 1..123: note = Synthetic polypeptide
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSS

SEQ ID NO: 29 - protein, 13 aa, synthetic construct
  REGION 1..13: note = Synthetic polypeptide
SGSSSNIGSNTVN

SEQ ID NO: 30 - protein, 8 aa, synthetic construct
  REGION 1..8: note = Synthetic polypeptide
YGNSNRPS

SEQ ID NO: 31 - protein, 11 aa, synthetic construct
  REGION 1..11: note = Synthetic polypeptide
QSYDSSLSGSV

SEQ ID NO: 32 - protein, 111 aa, synthetic construct
  REGION 1..111: note = Synthetic polypeptide
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLG

SEQ ID NO: 33 - protein, 5 aa, synthetic construct
  REGION 1..5: note = Synthetic polypeptide
SYWMY

SEQ ID NO: 34 - protein, 17 aa, synthetic construct
  REGION 1..17: note = Synthetic polypeptide
RIDPNSGSTKYNEKFKN

SEQ ID NO: 35 - protein, 11 aa, synthetic construct
  REGION 1..11: note = Synthetic polypeptide
DYRKGLYAMDY

SEQ ID NO: 36 - protein, 120 aa, synthetic construct
  REGION 1..120: note = Synthetic polypeptide
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 37 - protein, 11 aa, synthetic construct
  REGION 1..11: note = Synthetic polypeptide
KASQDVGTAVA

SEQ ID NO: 38 - protein, 7 aa, synthetic construct
  REGION 1..7: note = Synthetic polypeptide
WASTRHT

SEQ ID NO: 39 - protein, 9 aa, synthetic construct
  REGION 1..9: note = Synthetic polypeptide
QQYNSYPLT

SEQ ID NO: 40 - protein, 107 aa, synthetic construct
  REGION 1..107: note = Synthetic polypeptide
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 41 - protein, 5 aa, synthetic construct
  REGION 1..5: note = Synthetic polypeptide
TYAIS

SEQ ID NO: 42 - protein, 17 aa, synthetic construct
  REGION 1..17: note = Synthetic polypeptide
GIIPIFGKAHYAQKFQG

SEQ ID NO: 43 - protein, 14 aa, synthetic construct
  REGION 1..14: note = Synthetic polypeptide
KFHFVSGSPFGMDV

SEQ ID NO: 44 - protein, 123 aa, synthetic construct
  REGION 1..123: note = Synthetic polypeptide
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 45 - protein, 11 aa, synthetic construct
  REGION 1..11: note = Synthetic polypeptide
RASQSVSSYLA

SEQ ID NO: 46 - protein, 7 aa, synthetic construct
  REGION 1..7: note = Synthetic polypeptide
DASNRAT

SEQ ID NO: 47 - protein, 8 aa, synthetic construct
  REGION 1..8: note = Synthetic polypeptide
QQRSNWPT

SEQ ID NO: 48 - protein, 106 aa, synthetic construct
  REGION 1..106: note = Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 49-52 - skipped (4 entries)

SEQ ID NO: 53 - protein, 11 aa, synthetic construct
  REGION 1..11: note = Synthetic polypeptide
RAGQDVYKAVA

SEQ ID NO: 54 - protein, 8 aa, synthetic construct
  REGION 1..8: note = Synthetic polypeptide
YWSTNLYS

SEQ ID NO: 55 - protein, 8 aa, synthetic construct
  REGION 1..8: note = Synthetic polypeptide
GSTWPLTF

SEQ ID NO: 56 - protein, 107 aa, synthetic construct
  REGION 1..107: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQGSTWPLTFGQGTKVEIK

SEQ ID NO: 57-72 - skipped (16 entries)

SEQ ID NO: 73 - protein, 160 aa, synthetic construct
  REGION 1..160: note = Synthetic polypeptide
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 74 - protein, 15 aa, synthetic construct
  REGION 1..15: note = Synthetic polypeptide
LGGGGSGGGGSGGGG

SEQ ID NO: 75 - protein, 14 aa, synthetic construct
  REGION 1..14: note = Synthetic polypeptide
GGGGSGGGGSGGGG

SEQ ID NO: 76 - protein, 15 aa, synthetic construct
  REGION 1..15: note = Synthetic polypeptide
GGGGSGGGGSGGGGS

SEQ ID NO: 77 - protein, 20 aa, synthetic construct
  REGION 1..20: note = Synthetic polypeptide
GGGGSGGGGSGGGGSGGGGS

SEQ ID NO: 78 - protein, 25 aa, synthetic construct
  REGION 1..25: note = Synthetic polypeptide
GGGGSGGGGSGGGGSGGGGSGGGGS

SEQ ID NO: 79 - protein, 30 aa, synthetic construct
  REGION 1..30: note = Synthetic polypeptide
GGGGSGGGGSGGGGSGGGGSGGGGSGGGGS

SEQ ID NO: 80 - protein, 623 aa, synthetic construct
  REGION 1..623: note = Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 81 - protein, 625 aa, synthetic construct
  REGION 1..625: note = Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 82 - protein, 625 aa, synthetic construct
  REGION 1..625: note = Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 83 - protein, 622 aa, synthetic construct
  REGION 1..622: note = Synthetic polypeptide
EVQLVESGGGLVQPGGSLRLSCAASGFTITDSFIHWVRQAPGKGLEWVARIDPYGGTTNYADSVKGRFTISADTSKNTAYLQMNSLRAEDTAVYYCARAYSWFSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 84-91 - skipped (8 entries)

SEQ ID NO: 92 - protein, 13 aa, synthetic construct
  REGION 1..13: note = Synthetic polypeptide
AASGFTIDNTYIH

SEQ ID NO: 93 - skipped

SEQ ID NO: 94 - protein, 10 aa, synthetic construct
  REGION 1..10: note = Synthetic polypeptide
ARAFSWFPDY

SEQ ID NO: 95-108 - skipped (14 entries)

SEQ ID NO: 109 - protein, 10 aa, synthetic construct
  REGION 1..10: note = Synthetic polypeptide
RIDPANGKTT

SEQ ID NO: 110 - protein, 10 aa, synthetic construct
  REGION 1..10: note = Synthetic polypeptide
ARSFSWWADY

SEQ ID NO: 111 - protein, 117 aa, synthetic construct
  REGION 1..117: note = Synthetic polypeptide
EVQLVESGGGLVQPGGSLRLSCAASGFTIDNTYIHWVRQAPGKGLEWVARIDPANGKTTYADSVKGRFTISADTSKNTANLQMNSLRAEDTAVYYCARSFSWWADYWGQGTLVTVSS

SEQ ID NO: 112 - protein, 7 aa, synthetic construct
  REGION 1..7: note = Synthetic polypeptide
FNLQPTT

SEQ ID NO: 113 - protein, 107 aa, synthetic construct
  REGION 1..107: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQFNLQPTTFGQGTKVEIK

SEQ ID NO: 114 - protein, 10 aa, synthetic construct
  REGION 1..10: note = Synthetic polypeptide
RIDPANGVTR

SEQ ID NO: 115 - protein, 117 aa, synthetic construct
  REGION 1..117: note = Synthetic polypeptide
EVQLVESGGGLVQPGGSLRLSCAASGFTIDNTYIHWVRQAPGKRLEWVARIDPANGVTRYADSVKGRFTISADTSKNTAYLQMNSLRAEDTAVYYCARAFSWFPDYWGQGTLVTVSS

SEQ ID NO: 116 - protein, 8 aa, synthetic construct
  REGION 1..8: note = Synthetic polypeptide
YGIAPPTF

SEQ ID NO: 117 - protein, 107 aa, synthetic construct
  REGION 1..107: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQYGIAPPTFGQGTKVEIK

SEQ ID NO: 118-122 - skipped (5 entries)

SEQ ID NO: 123 - protein, 13 aa, synthetic construct
  REGION 1..13: note = Synthetic polypeptide
AASGFTIDNTYIH

SEQ ID NO: 124 - protein, 10 aa, synthetic construct
  REGION 1..10: note = Synthetic polypeptide
RIDPANGMTR

SEQ ID NO: 125 - protein, 117 aa, synthetic construct
  REGION 1..117: note = Synthetic polypeptide
EVQLVESGGGLVQPGGSLRLSCAASGFTIDNTYIHWVRQAPGKGLEWVARIDPANGMTRYADSVKGRFTISADTSKNTAYLQMNSLRAEDTAVYYCARAFSWFPDYWGQGTLVTVSS

SEQ ID NO: 126 - protein, 622 aa, synthetic construct
  REGION 1..622: note = Synthetic polypeptide
EVQLVESGGGLVQPGGSLRLSCAASGFTISDGYIHWVRQAPGKGLEWVARIDPLTGRTMYADSVKGRFTISADTSKNTAYLQMNSLRAEDTAVYYCARAFSWWPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 127 - protein, 214 aa, synthetic construct
  REGION 1..214: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQIMSPPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 622 aa, synthetic construct
  REGION 1..622: note = Synthetic polypeptide
EVQLVESGGGLVQPGGSLRLSCAASGFTIDNTYIHWVRQAPGKGLEWVARIDPVSGRTRYADSVKGRFTISADTSKNTADLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 129 - protein, 214 aa, synthetic construct
  REGION 1..214: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQFGATPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 622 aa, synthetic construct
  REGION 1..622: note = Synthetic polypeptide
EVQLVESGGGLVQPGGSLRLSCAASGFTISDAYIHWVRQAPGKGLEWVARIEPLSGRTDYADSVKGRFTISADTSKNTAYLQMNSLRAEDTAVYYCARAFSWFMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 131 - protein, 214 aa, synthetic construct
  REGION 1..214: note = Synthetic polypeptide
DIQMTQSPSSLSASVGDRVTITCRAGQDVYKAVAWYQQKPGKAPKLLIYWSTNLYSGVPSRFSGSGSGTDFTLMISSLQPEDFATYYCQQHDKTPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 622 aa, synthetic construct
  REGION 1..622: note = Synthetic polypeptide
EVQLVESGGGLVQPGGSLRLSCAASGFTIEDSYIHWVRQAPGKGLEWVARIDPITGLTHYADSVKGRFTISADTSKNTAYLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 133 - protein, 214 aa, synthetic construct
  REGION 1..214: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQIMEPPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 596 aa, synthetic construct
  REGION 1..596: note = Synthetic polypeptide
EVQLVESGGGLVQPGGSLRLSCAASGFTIDNTYIHWVRQAPGKGLEWVARIDPANGKTTYADSVKGRFTISADTSKNTANLQMNSLRAEDTAVYYCARSFSWWADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEK

SEQ ID NO: 135 - protein, 214 aa, synthetic construct
  REGION 1..214: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQFNLQPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 622 aa, synthetic construct
  REGION 1..622: note = Synthetic polypeptide
EVQLVESGGGLVQPGGSLRLSCAASGFTIDNTYIHWVRQAPGKRLEWVARIDPANGVTRYADSVKGRFTISADTSKNTAYLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 137 - protein, 214 aa, synthetic construct
  REGION 1..214: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQYGIAPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 622 aa, synthetic construct
  REGION 1..622: note = Synthetic polypeptide
EVQLVESGGGLVQPGGSLRLSCAASGFTITDGYIHWVRQAPGKGLEWVARIDPYNGRTNYADSVKGRFTISADTSKNTAYLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 139 - protein, 214 aa, synthetic construct
  REGION 1..214: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQHGNAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 622 aa, synthetic construct
  REGION 1..622: note = Synthetic polypeptide
EVQLVESGGGLVQPGGSLRLSCAASGFTIDNTYIHWVRQAPGKGLEWVARIDPANGMTRYADSVKGRFTISADTSKNTAYLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 141 - protein, 214 aa, synthetic construct
  REGION 1..214: note = Synthetic polypeptide
DIQMTQSPSSLSASVGDRVTITCRAGQDVYKAVAWYQQKPGKAPKLLIYWSTNLYSGVPSRFSGSGSGTDFTLTISSLQPEDFATYYCQQGSTWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - skipped

SEQ ID NO: 143 - protein, 214 aa, synthetic construct
  REGION 1..214: note = Synthetic polypeptide
DIQMTQSPSSLSASVGDRVTITCRASQDVSTAVAWQQKPGKAPKLLIYSASFLYSGVPSRFSGSGSGTDFTLTISSLQPEDFATYYCQQYLYHPATFGQGTKVEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - skipped

SEQ ID NO: 145 - protein, 216 aa, synthetic construct
  REGION 1..216: note = Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 146 - skipped

SEQ ID NO: 147 - protein, 215 aa, synthetic construct
  REGION 1..215: note = Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAWYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148-156 - skipped (9 entries)

SEQ ID NO: 157 - protein, 447 aa, synthetic construct
  REGION 1..447: note = Synthetic polypeptide
EVQLVESGGGLVQPGGSLRLSCAASGFTIDNTYIHWVRQAPGKGLEWVARIDPANGKTTYADSVKGRFTISADTSKNTANLQMNSLRAEDTAVYYCARSFSWWADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 447 aa, synthetic construct
  REGION 1..447: note = Synthetic polypeptide
EVQLVESGGGLVQPGGSLRLSCAASGFTIDNTYIHWVRQAPGKRLEWVARIDPANGVTRYADSVKGRFTISADTSKNTAYLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - skipped

SEQ ID NO: 160 - protein, 447 aa, synthetic construct
  REGION 1..447: note = Synthetic polypeptide
EVQLVESGGGLVQPGGSLRLSCAASGFTIDNTYIHWVRQAPGKGLEWVARIDPANGMTRYADSVKGRFTISADTSKNTAYLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161-171 - skipped (11 entries)

SEQ ID NO: 172 - protein, 160 aa, synthetic construct
  REGION 1..160: note = R104Q
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRRCHRFLPCENKSKAVEQVKNAFNKLQEKGIYKAMSEFDIFINYIEAYMTMKIRN

SEQ ID NO: 173 - protein, 160 aa, synthetic construct
  REGION 1..160: note = R107A
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRACHRFLPCENKSKAVEQVKNAFNKLQEKGIYKAMSEFDIFINYIEAYMTMKIRN

SEQ ID NO: 174 - protein, 160 aa, synthetic construct
  REGION 1..160: note = R107E
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRECHRFLPCENKSKAVEQVKNAFNKLQEKGIYKAMSEFDIFINYIEAYMTMKIRN

SEQ ID NO: 175 - protein, 160 aa, synthetic construct
  REGION 1..160: note = R107Q
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRQCHRFLPCENKSKAVEQVKNAFNKLQEKGIYKAMSEFDIFINYIEAYMTMKIRN

SEQ ID NO: 176 - protein, 160 aa, synthetic construct
  REGION 1..160: note = R107D
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRDCHRFLPCENKSKAVEQVKNAFNKLQEKGIYKAMSEFDIFINYIEAYMTMKIRN

SEQ ID NO: 177 - protein, 160 aa, synthetic construct
  REGION 1..160: note = R104Q/R107A
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRACHRFLPCENKSKAVEQVKNAFNKLQEKGIYKAMSEFDIFINYIEAYMTMKIRN

SEQ ID NO: 178 - protein, 160 aa, synthetic construct
  REGION 1..160: note = R104Q/R107E
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRECHRFLPCENKSKAVEQVKNAFNKLQEKGIYKAMSEFDIFINYIEAYMTMKIRN

SEQ ID NO: 179 - protein, 160 aa, synthetic construct
  REGION 1..160: note = R104Q/R107Q
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRQCHRFLPCENKSKAVEQVKNAFNKLQEKGIYKAMSEFDIFINYIEAYMTMKIRN

SEQ ID NO: 180 - protein, 160 aa, synthetic construct
  REGION 1..160: note = R104Q/R107D
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRDCHRFLPCENKSKAVEQVKNAFNKLQEKGIYKAMSEFDIFINYIEAYMTMKIRN

(19) United States
(12) Patent Application Publication
(10) Pub. No.: US 2024/0336667 A1
(43) Pub. Date: Oct. 10, 2024

(54) METHOD OF TREATING DISEASES WITH ANTI-PD-L1/IL-10 FUSION PROTEINS

(71) Applicant: ELIXIRON IMMUNOTHERAPEUTICS (HONG KONG) LIMITED, Hong Kong (CN)

(72) Inventors: Hung-Kai CHEN, Los Altos, CA (US); Pandelakis Andreas KONI, Larkspur, CA (US); Huey-Wen HSIAO, Taipei City (TW)

(21) Appl. No.: 18/700,015

(22) PCT Filed: Oct. 7, 2022
(86) PCT No.: PCT/US2022/077756

§ 371 Date: Apr. 10, 2024

Related U.S. Application Data
(60) Provisional application No. 63/254,116, filed on Oct. 10, 2021.

(51) Int. Cl. C07K 14/54; A61K 38/00; A61P 35/00; A61P 37/04

(52) U.S. Cl. CPC C07K 14/5428; A61P 35/00; A61P 37/04; A61K 38/00; C07K 2317/565; C07K 2319/30

(57) ABSTRACT

The present disclosure provides uses of with PD-L1 antibody-IL-10 fusion protein in the treatment of cancer and chronic virus infection diseases and the prevention of cancer recurrence via promoting T cell memory response.

### CROSS REFERENCE TO RELATED APPLICATIONS

This application claims the benefit under 35 U.S.C. § 119 (e) to U.S. Provisional Patent Application Ser. No. 63/254,116, filed on Oct. 10, 2021, the entirety of which is incorporated herein by reference. The Sequence Listing filed herewith, generated on Oct. 1, 2022, and filed in ST26 Extensible Markup Language format, named 2022-10-07T_256-004PCT_SEQ_List.xml, is 160 kilobyte, and is incorporated by reference in its entirety.

### FIELD OF THE INVENTION

The present disclosure relates generally to methods for treating disease with PD-L1 antibody-IL-10 fusion protein, particularly methods that use PD-L1 antibody-IL-10 fusion protein in the treatment of cancer and chronic virus infection diseases and the prevention of cancer recurrence via promoting T cell memory response.

### BACKGROUND OF THE INVENTION

Cancers represent a large group of diseases that involve abnormal cell growth with the potential to invade or spread to other parts of the body and constitute a primary cause of death. Because cancer cells are transformed (carcinogenesis) from normal cells, the antigenic surface proteins and/or glycoproteins presented by cancer cells are identical to or are highly similar to antigens that are present on normal, non-tumor cells in the host organism. The host organism's immune system therefore can have difficulty detecting and distinguishing cancer cells from normal cells. Additionally, cancer cells can adopt another mechanism to avoid host immune system detection.

Programmed death-ligand 1 (PD-L1) is a transmembrane protein that binds to the inhibitory checkpoint molecule, PD1 and thereby suppresses the adaptive immune response during pregnancy, autoimmune disease, and other disease states, such as hepatitis. Additionally, PD-L1 is highly expressed in cancer tissue and its level of expression has been found to correlate strongly with tumor aggressiveness. The over-expression of PD-L1 and its binding to its receptor protein, PD1 is believed to be critical to the mechanism by which cancer cells avoid destruction by the immune system of the host organism.

Interleukin 10 or “IL-10” (also known as cytokine synthesis inhibitory factor, CSIF, IL-10, IL10A, GVHDS, or TGIF) is a cytokine that has multiple effects in immunoregulation and inflammation. IL-10 is known to downregulate the expression of Th1 cytokines, MHC class II antigens, and co-stimulatory molecules on macrophages. IL-10 is also known to enhance B cell survival, proliferation, and antibody production. IL-10 can block NF-κB activity and is involved in the regulation of the JAK-STAT signaling pathway. IL-10 is capable of inhibiting synthesis of pro-inflammatory cytokines such as IFN-γ, IL-2, IL-3, TNFα and GM-CSF made by cells such as macrophages and Th1 T cells. It also displays a potent ability to suppress the antigen-presentation capacity of antigen presenting cells; however, it is also stimulatory towards certain T cells (Th2) and mast cells and stimulates B cell maturation and antibody production.

IL-10 has been recognized as a potential inhibitor of tumor metastasis and an immunostimulatory agent useful in immuno-oncology treatments. In transgenic mice expression of IL-10 or dosing with IL-10 have been observed to control of primary tumor growth and decrease metastatic burden. A PEGylated version of recombinant murine IL-10 has been shown to induce IFNγ and CD8+ T cell dependent anti-tumor immunity in mouse models. PEGylated recombinant human IL-10 has been shown to enhance CD8+ T cell secretion of the cytotoxic molecules Granzyme B and Perforin and potentiate T cell receptor dependent IFNγ secretion. In clinical trials the PEGylated recombinant human IL-10 (PEG-rHuIL-10, AM0010) has been found to exhibit substantial anti-tumor efficacy, eliciting a dose titratable induction of the immune stimulatory cytokines IFNγ, IL-18, IL-7, GM-CSF, and IL-4. Treated patients also exhibited an increase of peripheral CD8+ T cells expressing markers of activation, such as PD1, lymphocyte activation gene 3 (LAG3)+ and increased Fas Ligand (FasL), and a decrease in serum TGFβ. These findings suggest that IL-10 treatment results in a predominantly immunostimulatory effect in humans.

### SUMMARY OF THE INVENTION

In contrast to the understanding in the art regarding anti-PD-L1/IL-10 fusion proteins, the present disclosure provides compositions and use of the compositions for treating cancer, preventing cancer recurrence and treating chronic virus infection diseases based upon the surprising discovery that the anti-PD-L1/IL-10 fusion proteins capable of promoting T cell memory response. Without being bound by theory, it is proposed that anti-PD-L1/IL-10 fusion proteins lead to durable memory responses via progenitor exhausted T cells. In a patient with cancer or chronic virus infection, it is proposed that anti-PD-L1/IL-10 fusion proteins lead to T cell memory response that treats cancer or virus infection and prevents cancer recurrence or chronic virus infection. Accordingly, the present disclosure provides methods of treatment wherein the patient with cancer or chronic virus infection is administered a PD-L1 antibody-IL-10 fusion protein, wherein the PD-L1 antibody-IL-10 fusion protein promotes durable memory responses via progenitor exhausted T cells.

In some embodiments, the present disclosure provides a use of a composition comprising a therapeutically effective amount of a PD-L1 antibody-IL-10 fusion protein and a pharmaceutically acceptable carrier for promoting T cell memory response in a subject in need thereof. In some embodiments, the present disclosure provides a use of a composition comprising a therapeutically effective amount of a PD-L1 antibody-IL-10 fusion protein and a pharmaceutically acceptable carrier for manufacture of a medicament for promoting T cell memory response in a subject in need thereof. In some embodiments, the present disclosure provides a composition comprising a therapeutically effective amount of a PD-L1 antibody-IL-10 fusion protein and a pharmaceutically acceptable carrier for use in promoting T cell memory response in a subject in need thereof.

In some embodiments, the present disclosure provides a use of a composition comprising a therapeutically effective amount of a PD-L1 antibody-IL-10 fusion protein and a pharmaceutically acceptable carrier for treating cancer in a subject in need thereof. In some embodiments, the present disclosure provides a use of a composition comprising a therapeutically effective amount of a PD-L1 antibody-IL-10 fusion protein and a pharmaceutically acceptable carrier for manufacture of a medicament for treating cancer in a subject in need thereof. In some embodiments, the present disclosure provides a composition comprising a therapeutically effective amount of a PD-L1 antibody-IL-10 fusion protein and a pharmaceutically acceptable carrier for use in treating cancer in a subject in need thereof.

In some embodiments, the present disclosure provides a use of a composition comprising a therapeutically effective amount of a PD-L1 antibody-IL-10 fusion protein and a pharmaceutically acceptable carrier for preventing cancer recurrence in a subject in need thereof. In some embodiments, the present disclosure provides a use of a composition comprising a therapeutically effective amount of a PD-L1 antibody-IL-10 fusion protein and a pharmaceutically acceptable carrier for manufacture of a medicament for preventing cancer recurrence in a subject in need thereof. In some embodiments, the present disclosure provides a composition comprising a therapeutically effective amount of a PD-L1 antibody-IL-10 fusion protein and a pharmaceutically acceptable carrier for use in preventing cancer recurrence in a subject in need thereof.

In some embodiments, the present disclosure provides a use of a composition comprising a therapeutically effective amount of a PD-L1 antibody-IL-10 fusion protein and a pharmaceutically acceptable carrier for treating chronic virus infection diseases in a subject in need thereof. In some embodiments, the present disclosure provides a use of a composition comprising a therapeutically effective amount of a PD-L1 antibody-IL-10 fusion protein and a pharmaceutically acceptable carrier for manufacture of a medicament for treating chronic virus infection diseases in a subject in need thereof. In some embodiments, the present disclosure provides a composition comprising a therapeutically effective amount of a PD-L1 antibody-IL-10 fusion protein and a pharmaceutically acceptable carrier for use in treating chronic virus infection diseases in a subject in need thereof.

In some embodiments, the composition is for use with at least one additional therapeutic agent. In other embodiments, the at least one additional therapeutic agent is a virus vaccine. In other embodiments, the at least one additional therapeutic agent is an antibody that targets an inhibitory immune checkpoint molecule. In some preferred embodiments, the at least one additional therapeutic agent is selected from the group consisting of: an imaging agent; a cytotoxic agent; an angiogenesis inhibitor; a kinase inhibitor; a co-stimulation molecule blocker; an adhesion molecule blockers; an anti-cytokine antibody or functional fragment thereof; methotrexate; cyclosporin; rapamycin; FK506; a detectable label or reporter; a TNF antagonist; an anti-rheumatic; a muscle relaxant; a narcotic; a non-steroid anti-inflammatory drug (NSAID); an analgesic; an anesthetic; a sedative; a local anesthetic; a neuromuscular blocker; an antimicrobial; an antipsoriatic; a corticosteroid; an anabolic steroid; an erythropoietin; an immunization; an immunoglobulin; an immunosuppressive; a growth hormone; a hormone replacement drug; a radiopharmaceutical; an antidepressant; an antipsychotic; a stimulant; an asthma medication; a beta agonist; an inhaled steroid; an epinephrine or analog thereof; a cytokine; a cytokine antagonist; an immunomodulatory agent; an HBV entry inhibitor; a viral RNA inhibitor; a gene editing agent; an HBsAg secretion inhibitor; a polymerase inhibitor; an interferon; a viral entry inhibitor; a viral maturation inhibitor; a nucleoside reverse transcriptase inhibitor; a capsid assembly inhibitor/modulator; a cccDNA inhibitor; an FXR agonist; a microRNA; and a TLR agonist.

In some embodiments, the composition is for administration by at least one mode selected from the group consisting of: parenteral, subcutaneous, intramuscular, intravenous, intra-articular, intrabronchial, intraabdominal, intracapsular, intracavitary, intracartilaginous, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal. In some embodiments, the composition is for use more than once a day, at least once a day, at least once a week, or at least once a month.

In some embodiments, the PD-L1 antibody is an immunoglobulin molecule, an Fv, a disulfide linked Fv, a monoclonal antibody, an scFv, a chimeric antibody, a single domain antibody, a CDR-grafted antibody, a diabody, a human antibody, a humanized antibody, a multispecific antibody, an Fab, a dual specific antibody, an Fab′ fragment, a bispecific antibody, an F(ab′)2 fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody, a dAb fragment, an isolated complementarity determining region (CDR), or a single chain antibody. In some embodiments, the PD-L1 antibody comprises a heavy chain (HC) fused via a linker to the IL10; optionally, wherein the linker comprises an amino acid sequence selected from SEQ ID NO: 74, 75, 76, 77, 78, and 79.

In some embodiments, (a) the IL-10 comprises an amino acid sequence of SEQ ID NO: 73, 172, 173, 174, 175, 176, 177, 178, 179 or 180; (b) the IL-10 is a naturally-occurring or engineered variant of IL-10 that retains its cytokine activity; (c) the IL-10 is a synthetically modified version of IL-10 that retains its cytokine activity; and/or (d) the IL-10 comprises one, two, or four IL-10 polypeptides.

In some embodiments, the IL-10 comprises a substitution on amino acids in position 104, position 107, and a combination thereof, relative to amino acids of wild-type IL-10. In some embodiments, the wild-type IL-10 comprises the amino acid sequence having at least 80%, preferably at least 90%, and more preferably at least 95%, identity with SEQ ID NO: 73, 172, 173, 174, 175, 176, 177, 178, 179 or 180. In some embodiments, the substitution comprises: (a) R104Q; (b) any one of R107A, R107E, R107Q and R107D; or (c) a combination thereof. In some embodiments, the substitution comprises R104Q/R107A, R104Q/R107E, R104Q/R107Q or R104Q/R107D.

In some embodiments, the PD-L1 antibody comprises (i) a first light chain complementary determining region (CDR-L1), a second light chain complementary determining region (CDR-L2), and a third light chain complementary determining region (CDR-L3), and (ii) a first heavy chain complementary determining region (CDR-H1), a second heavy chain complementary determining region (CDR-H2), and a third heavy chain complementary determining region (CDR-H3), wherein:
(a) CDR-H1 comprises an amino acid sequence selected from SEQ ID NO: 92, and 123;
(b) CDR-H2 comprises an amino acid sequence selected from SEQ ID NO: 109, 114, and 124;
(c) CDR-H3 comprises an amino acid sequence selected from SEQ ID NO: 94, and 110;
(d) CDR-L1 comprises an amino acid sequence selected from SEQ ID NO: 53;
(e) CDR-L2 comprises an amino acid sequence selected from SEQ ID NO: 54;
(f) CDR-L3 comprises an amino acid sequence selected from SEQ ID NO: 55, 112, and 116.

In some embodiments,
(a) CDR-H1 comprises the amino acid sequence of SEQ ID NO: 92, CDR-H2 comprises the amino acid sequence of SEQ ID NO: 109, CDR-H3 comprises the amino acid sequence of SEQ ID NO: 110, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 112;
(b) CDR-H1 comprises the amino acid sequence of SEQ ID NO: 92, CDR-H2 comprises the amino acid sequence of SEQ ID NO: 114, CDR-H3 comprises the amino acid sequence of SEQ ID NO: 94, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 116; or
(c) CDR-H1 comprises the amino acid sequence of SEQ ID NO: 123, CDR-H2 comprises the amino acid sequence of SEQ ID NO: 124, CDR-H3 comprises the amino acid sequence of SEQ ID NO: 95, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 55.

In some embodiments, the antibody comprises a heavy chain variable domain (VH) amino acid sequence having at least 90% identity to a sequence selected from SEQ ID NO: 111, 115, and 125; and/or a light chain variable domain (VL) amino acid sequence having at least 90% identity to a sequence selected from SEQ ID NO: 56, 113, and 117; optionally, wherein:
(a) the antibody comprises a VH amino acid sequence having at least 90% identity to SEQ ID NO: 111; and a VL amino acid sequence having at least 90% identity to SEQ ID NO: 113;
(b) the antibody comprises a VH amino acid sequence having at least 90% identity to SEQ ID NO: 115; and a VL amino acid sequence having at least 90% identity to SEQ ID NO: 117; or
(c) the antibody comprises a VH amino acid sequence having at least 90% identity to SEQ ID NO: 125; and a VL amino acid sequence having at least 90% identity to SEQ ID NO: 56.

In some embodiments, the antibody comprises a heavy chain (HC) amino acid sequence having at least 90% identity to a sequence selected from SEQ ID NO: 157, 158, and 160, and/or a light chain (LC) amino acid sequence having at least 90% identity to a sequence selected from SEQ ID NO: 135, 137, and 141; optionally, wherein the antibody comprises:
(a) the HC amino acid sequence of SEQ ID NO: 157, and the LC amino acid sequence of SEQ ID NO: 135;
(b) the HC amino acid sequence of SEQ ID NO: 158, and the LC amino acid sequence of SEQ ID NO: 137;
(c) the HC amino acid sequence of SEQ ID NO: 160, and the LC amino acid sequence of SEQ ID NO: 141.

In some embodiments, the antibody comprises a heavy chain (HC) fused via a linker to an IL-10 polypeptide, wherein the HC-IL-10 fusion amino acid sequence has at least 90% identity to a sequence selected from SEQ ID NO: 134, 136, and 140; and a light chain (LC) having an amino acid sequence of at least 90% identity to a sequence selected from SEQ ID NO: 135, 137, and 141; optionally, wherein the antibody comprises:
(a) the HC-IL-10 fusion amino acid sequence of SEQ ID NO: 134, and the LC amino acid sequence of SEQ ID NO: 135;
(b) the HC-IL-10 fusion amino acid sequence of SEQ ID NO: 136, and the LC amino acid sequence of SEQ ID NO: 137; or
(d) the HC-IL-10 fusion amino acid sequence of SEQ ID NO: 140, and the LC amino acid sequence of SEQ ID NO: 141.

In some embodiments, the PD-L1 antibody comprises a heavy chain (HC) fused via a linker to the IL10, wherein the antibody comprises (i) a first light chain complementary determining region (CDR-L1), a second light chain complementary determining region (CDR-L2), and a third light chain complementary determining region (CDR-L3), and (ii) a first heavy chain complementary determining region (CDR-H1), a second heavy chain complementary determining region (CDR-H2), and a third heavy chain complementary determining region (CDR-H3), wherein:
(a) CDR-H1 comprises the amino acid sequence of SEQ ID NO: 1, CDR-H2 comprises the amino acid sequence of SEQ ID NO: 2, CDR-H3 comprises the amino acid sequence of SEQ ID NO: 3, CDR-L1 comprises the amino acid sequence of SEQ ID NO: 5, CDR-L2 comprises the amino acid sequence of SEQ ID NO: 6, and CDR-L3 comprises the amino acid sequence of SEQ ID NO: 7;
(b) CDR-H1 comprises the amino acid sequence of SEQ ID NO: 9, CDR-H2 comprises the amino acid sequence of SEQ ID NO: 10, CDR-H3 comprises the amino acid sequence of SEQ ID NO: 11, CDR-L1 comprises the amino acid sequence of SEQ ID NO: 13, CDR-L2 comprises the amino acid sequence of SEQ ID NO: 14, and CDR-L3 comprises the amino acid sequence of SEQ ID NO: 15;
(c) CDR-H1 comprises the amino acid sequence of SEQ ID NO: 17, CDR-H2 comprises the amino acid sequence of SEQ ID NO: 18, CDR-H3 comprises the amino acid sequence of SEQ ID NO: 19, CDR-L1 comprises the amino acid sequence of SEQ ID NO: 21, CDR-L2 comprises the amino acid sequence of SEQ ID NO: 22, and CDR-L3 comprises the amino acid sequence of SEQ ID NO: 23;
(d) CDR-H1 comprises the amino acid sequence of SEQ ID NO: 25, CDR-H2 comprises the amino acid sequence of SEQ ID NO: 26, CDR-H3 comprises the amino acid sequence of SEQ ID NO: 27, CDR-L1 comprises the amino acid sequence of SEQ ID NO: 29, CDR-L2 comprises the amino acid sequence of SEQ ID NO: 30, and CDR-L3 comprises the amino acid sequence of SEQ ID NO: 31;
(e) CDR-H1 comprises the amino acid sequence of SEQ ID NO: 33, CDR-H2 comprises the amino acid sequence of SEQ ID NO: 34, CDR-H3 comprises the amino acid sequence of SEQ ID NO: 35, CDR-L1 comprises the amino acid sequence of SEQ ID NO: 37, CDR-L2 comprises the amino acid sequence of SEQ ID NO: 38, and CDR-L3 comprises the amino acid sequence of SEQ ID NO: 39;
(f) CDR-H1 comprises the amino acid sequence of SEQ ID NO: 41, CDR-H2 comprises the amino acid sequence of SEQ ID NO: 42, CDR-H3 comprises the amino acid sequence of SEQ ID NO: 43, CDR-L1 comprises the amino acid sequence of SEQ ID NO: 45, CDR-L2 comprises the amino acid sequence of SEQ ID NO: 46, and CDR-L3 comprises the amino acid sequence of SEQ ID NO: 47;
(g) CDR-H1 comprises the amino acid sequence of SEQ ID NO: 92, CDR-H2 comprises the amino acid sequence of SEQ ID NO: 109, CDR-H3 comprises the amino acid sequence of SEQ ID NO: 110, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 112;
(h) CDR-H1 comprises the amino acid sequence of SEQ ID NO: 92, CDR-H2 comprises the amino acid sequence of SEQ ID NO: 114, CDR-H3 comprises the amino acid sequence of SEQ ID NO: 94, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 116; or
(i) CDR-H1 comprises the amino acid sequence of SEQ ID NO: 123, CDR-H2 comprises the amino acid sequence of SEQ ID NO: 124, CDR-H3 comprises the amino acid sequence of SEQ ID NO: 94, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 55.

In some embodiments, the antibody comprises a heavy chain variable domain (VH) amino acid sequence having at least 90% identity to a sequence selected from SEQ ID NO: 4, 12, 20, 28, 36, 44, 111, 115, and 125; and/or a light chain variable domain (VL) amino acid sequence having at least 90% identity to a sequence selected from SEQ ID NO: 8, 16, 24, 32, 40, 48, 56, 113, and 117; optionally, wherein:
(a) the antibody comprises a VH amino acid sequence having at least 90% identity to SEQ ID NO: 4; and a VL amino acid sequence having at least 90% identity to SEQ ID NO: 8;
(b) the antibody comprises a VH amino acid sequence having at least 90% identity to SEQ ID NO: 12; and a VL amino acid sequence having at least 90% identity to SEQ ID NO: 16;
(c) the antibody comprises a VH amino acid sequence having at least 90% identity to SEQ ID NO: 20; and a VL amino acid sequence having at least 90% identity to SEQ ID NO: 24;
(d) the antibody comprises a VH amino acid sequence having at least 90% identity to SEQ ID NO: 28; and a VL amino acid sequence having at least 90% identity to SEQ ID NO: 32;
(e) the antibody comprises a VH amino acid sequence having at least 90% identity to SEQ ID NO: 36; and a VL amino acid sequence having at least 90% identity to SEQ ID NO: 40;
(f) the antibody comprises a VH amino acid sequence having at least 90% identity to SEQ ID NO: 44; and a VL amino acid sequence having at least 90% identity to SEQ ID NO: 48;
(g) the antibody comprises a VH amino acid sequence having at least 90% identity to SEQ ID NO: 111; and a VL amino acid sequence having at least 90% identity to SEQ ID NO: 113;
(h) the antibody comprises a VH amino acid sequence having at least 90% identity to SEQ ID NO: 115; and a VL amino acid sequence having at least 90% identity to SEQ ID NO: 117; or
(i) the antibody comprises a VH amino acid sequence having at least 90% identity to SEQ ID NO: 125; and a VL amino acid sequence having at least 90% identity to SEQ ID NO: 56.

In some embodiments, the antibody comprises a HC-IL-10 fusion amino acid sequence having at least 90% identity to a sequence selected from SEQ ID NO: 80, 81, 82, 134, 136, and 140, and a light chain (LC) amino acid sequence having at least 90% identity to a sequence selected from SEQ ID NO: 135, 137, 141, 143, 145, and 147; optionally, wherein
(a) the HC-IL-10 fusion amino acid sequence of SEQ ID NO: 80, and the LC amino acid sequence of SEQ ID NO: 143;
(b) the HC-IL-10 fusion amino acid sequence of SEQ ID NO: 81, and the LC amino acid sequence of SEQ ID NO: 145;
(c) the HC-IL-10 fusion amino acid sequence of SEQ ID NO: 82, and the LC amino acid sequence of SEQ ID NO: 147;
(d) the HC-IL-10 fusion amino acid sequence of SEQ ID NO: 134, and the LC amino acid sequence of SEQ ID NO: 135;
(e) the HC-IL-10 fusion amino acid sequence of SEQ ID NO: 136, and the LC amino acid sequence of SEQ ID NO: 137; or
(f) the HC-IL-10 fusion amino acid sequence of SEQ ID NO: 140, and the LC amino acid sequence of SEQ ID NO: 141.

In some embodiments, wherein:
(a) the protein increases MC/9 cell proliferation by at least 25%, at least 50%, at least 100%, at least 150%, at least 200% or more;
(b) the protein increases IFNγ and granzyme B production from activated CD8 T cells by at least 25%, at least 50%, at least 100%, or more; and/or
(c) the antibody decreases tumor volume in a syngeneic mouse tumor model measured at 28 days by at least 25%, at least 50%, at least 75%, or more, wherein the mouse tumor model is selected from: CT26 colon cancer and EMT6 breast cancer.

### BRIEF DESCRIPTION OF THE FIGURES

FIG. 1 illustrate that anti-PDL1/IL-10 fusion proteins enhance T-cell activation in mix lymphocyte reaction (MLR). Human CD4 T cells were isolated from PBMC and co-cultured with allogeneic mature DC in the presence of anti-PDL1/IL-10 fusion proteins (Avelumab/IL10, HSYPP411C/IL10, YP7G/IL10, YP11F/IL10, YT7A/IL10, YT7H/IL10 and YT10H/IL10). The level of IFNγ in supernatants were measured by ELISA (Biolegend) at day5.

FIG. 2 illustrate accumulation of anti-PDL1/IL-10 fusion protein in the tumor. (A) in vivo whole body and (B) ex vivo fluorescence images of spleen and tumor from Head and neck squamous cell carcinomas HNSC/Q1-luc tumor-bearing mice 24 hours after intravenous injection of vivoTag680-labeled anti-PDL1/IL-10 (150 μg).

FIG. 3 illustrate that anti-PDL1/IL-10 enhances T cell response. Syngeneic colorectal carcinoma CT26 tumor-bearing mice (n=7) were randomized 7 days post-implantation and then treated with IL10-Fc (3 mg/kg), anti-PDL1/IL-10 (6 mg/kg), or anti-PDL1/TGFbR (6 mg/kg) twice weekly for 3 weeks. Serum level of (A) IL-18 and (B) CXCL9 after treatment (Day29) were measured by ELISA (Biolegend). Mean±SD is shown. *p<0.05, **p<0.01, ***p<0.001

FIG. 4 illustrate that the ability of anti-PDL1/IL-10 to enhance tumor control was dependent on increased activity of tumor-resident cells. Co-treatment with FTY720, which inhibits lymphocyte egress from secondary lymphoid organs, did not reduce anti-tumor activity in some albeit not all mice. Syngeneic breast carcinoma EMT6 tumor-bearing mice (n=6) were randomized on day 10 and treated with vehicle (1% ethanol) or FTY720 (2 mg/kg, PO) daily for 2 weeks. Anti-PDL1 (10 mg/kg) or anti-PDL1/IL-10 (12 mg/kg) were administered twice weekly from day 11 for 4 weeks. (A) Tumor growth was measured twice weekly. (B) survival curves. Mean±SEM is shown. *p<0.05, **p<0.01, ***p<0.001

FIG. 5 illustrate that anti-PDL1/IL-10 treatment allowed for subsequent, secondary tumor rejection in mice ‘cured’ of their primary tumor, indicating durable T cell memory protection. Syngeneic EMT6 tumor-bearing mice (n=7) were randomized 7 days post-implantation and treated with anti-PDL1 (5 mg/kg), anti-PDL1/IL-10 (6 mg/kg) or anti-PDL1/TGFbR (6 mg/kg) twice weekly for 3 weeks. (A) Survival curves for primary tumor study. For rechallenge study, 5 weeks after tumor ‘cure’ (day 67), cured and naïve Balb/c mice (n=5) were implanted with EMT6 tumor cells and (B) tumor growth monitored for 4 weeks. Mean±SEM is shown. *p<0.05, **p<0.01, ***p<0.001

FIG. 6 illustrate that anti-PDL1/IL-10 mutants control tumor growth and enhance T cell response. Syngeneic colorectal carcinoma CT26 tumor-bearing mice were randomized 7 days post-implantation and then treated with 1 mg/kg anti-PDL1/IL-10 or anti-PDL1/IL-10 mutants (R107A, R104Q/R107A) twice weekly for 3 weeks. (A) Tumor growth was measured twice weekly. (B) Serum level of CXCL9 after treatment (Day28) was measured by ELISA (Biolegend). Mean+SD is shown. *p<0.05, **p<0.01.

FIG. 7 illustrate that tumor-infiltrating lymphocytes (TILs) isolated from anti-PDL1/IL10-treated tumor-bearing mice exhibit better recall responses. C57BL/6 mice were inoculated with YUMM1.7-GP33 melanoma cells and received i.v. adoptive cell transfer of early activated Tcf7^{GFP} P14 T cells on Day 9, followed by treatments of PBS or anti-PDL1/IL-10 on Day 10 and Day 14. PEX population was further sorted out and transffered into naïve recipients for LCMV-Arm rechallenge. Upon rechallenge, TILs treated with anti-PDL1/IL-10 display better immune qualities with decreased expression level of co-inhibitory receptors, (A) PD1 and (B) TIM3, and (C) decreased PD-1⁺TIM3⁺LAG3⁺ cell population in comparison with PBS-treated group. PEX, progenitor exhausted T cells.

FIG. 8 illustrate that anti-PDL1/IL-10 treatment boosts PEX-dependent durable tumor protection. C57BL/6 mice were inoculated subcutaneously with YUMM1.7-GP33 tumor cells on Day 0 and received i.v. adoptive transfer of early activated Tcf7^{DTR-GFP} P14 T cells on Day 6. Tumor-bearing mice were further i.p. administrated with anti-PDL1/IL-10 every three days starting on Day 8 until tumors became undetectable. Three weeks later, tumor-free mice were either i.p. injected three times with PBS or DT to eliminate TCF1+ DTR-expressing cells within 1 week, followed by s.c. inoculation with B16-GP33 cells. Age-matched naïve mice were inoculated with B16-GP33 melanoma cells as a positive control (Naïve) for tumor progression. (A) Tumor growth and (B) tumor-free survival were monitored for 4 weeks. The control group (Ctrl) of tumor-free mice without elimination of PEX remains tumor-free status, whereas B16-GP33 tumor lesions can be observed on Day 14-16 post rechallenge in DT-treated group, indicating that anti-PDL1/IL10-induced durable protection is PEX-dependent. PEX, progenitor exhausted T cells.

### DETAILED DESCRIPTION

The present disclosure provides methods of treatment and associated compositions based upon the surprising discovery that anti-PD-L1/IL-10 fusion proteins capable of promoting T cell memory response are useful in treating cancer, preventing cancer recurrence and treating virus infection diseases, preferably chronic virus infection. Accordingly, the present disclosure provides methods of treatment of cancer, cancer recurrence and virus infection wherein a patient in need thereof is administered an anti-PD-L1/IL-10 fusion proteins. The anti-PD-L1/IL-10 fusion proteins useful in the methods and compositions are capable of inducing, enhancing and promoting T cell memory response. As described in greater detail below, the methods of treatment and associated compositions are thus capable of stimulating and/or otherwise restoring normal immune function that can effectively treating cancer, preventing cancer recurrence and treating virus infection from the subject in need.

### Terminology and Techniques

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”); Current Protocols in Molecular Biology, F. M. Ausubel et al., eds., Current Protocols, a joint venture between Greene Publishing Associates, Inc. and John Wiley & Sons, Inc. (supplemented through 2011) (hereinafter “Ausubel”); Antibody Engineering, Vols. 1 and 2, R. Kontermann and S. Dubel, eds., Springer-Verlag, Berlin and Heidelberg (2010); Monoclonal Antibodies: Methods and Protocols, V. Ossipow and N. Fischer, eds., 2nd Ed., Humana Press (2014); Therapeutic Antibodies: From Bench to Clinic, Z. An, ed., J. Wiley & Sons, Hoboken, N.J. (2009); and Phage Display, Tim Clackson and Henry B. Lowman, eds., Oxford University Press, United Kingdom (2004).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

“PD-L1” (or “PDL1”), as used herein, refers to the transmembrane protein, programmed death-ligand 1, and as used herein encompasses the PD-L1 proteins of human, cynomolgus monkey, mouse, and any isoforms of these proteins. Amino acid sequences of various exemplary PD-L1 proteins are known in the art and are provided in Table 2 below and the attached Sequence Listing.

“IL10” or “IL-10,” as used herein, refers to the cytokine, interleukin 10, also known as cytokine synthesis inhibitory factor (CSIF), and is intended to also include naturally-occurring variants, engineered variants, and/or synthetically modified versions of interleukin 10 that retain its cytokine functions. Amino acid sequences of various exemplary IL-10 polypeptides and recombinant IL-10 fusion constructs are provided in Table 1 below and the attached Sequence Listing. Other exemplary engineered and/or modified IL-10 polypeptides that retain cytokine functions are known in the art (see e.g., U.S. Pat. No. 7,749,490 B2; US 2017/0015747 A1; Naing, A. et al. “PEGylated IL-10 (Pegilodecakin) Induces Systemic Immune Activation, CD8+ T Cell Invigoration and Polyclonal T Cell Expansion in Cancer Patients.” Cancer Cell 34, 775-791.e3 (2018); Gorby, C. et al. “Engineered IL-10 variants elicit potent immunomodulatory effects at low ligand doses.” Sci Signal 13, (2020); Yoon, S. I. et al. “Epstein-Barr virus IL-10 engages IL-10R1 by a two-step mechanism leading to altered signaling properties.” J Biol Chem 287, 26586-26595 (2012)).

“Fusion protein,” as used herein, refers to two or more protein and/or polypeptide molecules that are linked (or “fused”) in a configuration that does not occur naturally. An exemplary fusion protein of the present disclosure includes the “IL10-Fc” fusion protein that comprises an IL10 polypeptide covalently linked through a polypeptide linker sequence at its C-terminus to an immunoglobulin Fc region polypeptide. Fusion proteins of the present disclosure also include “antibody fusions” that an antibody covalently conjugated (or fused) to a polypeptide or protein, typically via a linker to a terminus of the antibody's light chain (LC) or heavy chain (HC). Exemplary antibody fusions of the present disclosure include an anti-PD-L1 antibody fused to a recombinant IL10 polypeptide via a 15 amino acid polypeptide linker (e.g., SEQ ID NO: 74) from the C-terminus of the antibody heavy chain to the N-terminus of the IL10 polypeptide. Antibody fusions are labeled herein with an “antibody/polypeptide” nomenclature to indicate the fusion components, such as “Ab/IL10” or “anti-PD-L1/IL10.” As described elsewhere herein, an antibody fusion of the present disclosure can include a full-length IgG antibody, comprising a dimeric complex of heavy chain-light chain pairs, where each heavy chain C-terminus is linked through a polypeptide linker sequence to an IL10 polypeptide. Amino acid sequences of various exemplary fusion proteins are provided in Table 2 below and the attached Sequence Listing.

“Polypeptide linker” or “linker sequence” as used herein refers to a chain of two or more amino acids with each end of the chain covalently attached to a different polypeptide molecule, thereby functioning to conjugate or fuse the different polypeptides. Typically, polypeptide linkers comprise polypeptide chains of 5 to 30 amino acids. A wide range of polypeptide linkers are known in the art and can be used in the compositions and methods of the present disclosure. Exemplary polypeptide linkers include in the compositions and methods of the present disclosure include, (GGGGS)n, (SSSSG)n, (GGGG)(SGGGG)n, (EAAAK)n, (XP)n, ENLYFQ(-G/S), typically, where n is 1 to 6, and other specific linker sequences as disclosed elsewhere herein.

“Anti-PD-L1 antibody” or “antibody that binds PD-L1” refers to an antibody that binds PD-L1 with sufficient affinity such that the antibody is useful as a therapeutic and/or diagnostic agent for targeting PD-L1. In some embodiments, the extent of binding of an anti-PD-L1 specific antibody to an unrelated, non-PD-L1 antigen is less than about 20%, less than about 15%, less than about 10%, or less than about 5% of the binding of the antibody to PD-L1 as measured by, e.g., radioimmunoassay (RIA) or surface plasmon resonance (SPR). In some embodiments, an anti-PD-L1 antibody of the present disclosure has a dissociation constant (K_{D}) of <1 μM, <100 nM, <10 nM, <1 nM, <0.1 nM, <0.01 nM, or <1 pM (e.g., 10⁻⁸ M or less, e.g., from 10⁻⁸ M to 10⁻¹³ M, e.g., from 10⁻⁹ M to 10⁻¹³ M).

“T cells memory response,” as used herein, refers to activation of T cells after the T cells have previously encountered and responded to their specific antigen, or after T cells have differentiated from activated T cells. For example, although tumor-specific memory T cells make up a small portion of the total T cell amount, they play an important function in surveillance of tumor cells during a person's entire lifespan. In a case where tumor-specific memory T cells encounter tumor cells that express their specific tumor antigen, the memory T cells are immediately activated and clonally expanded. The activated and expanded T cells differentiate into effector T cells to kill tumor cells with high efficiency. Memory T cells are important for establishing and maintaining long-term tumor antigen-specific responses of T cells. In the present invention, activated T cells with memory response specifically recognize antigens on cancer cells, so that such T cells can treat a cancerous or neoplastic condition or prevent recurrence, progression, or metastasis of cancer while avoiding the defense mechanism of cancer cells.

“Antibody,” as used herein, refers to a molecule comprising one or more polypeptide chains that specifically binds to, or is immunologically reactive with, a particular antigen. Exemplary antibodies of the present disclosure include native antibodies, whole antibodies, monoclonal antibodies, polyclonal antibodies, chimeric antibodies, humanized antibodies, human antibodies, multispecific (or heteroconjugate) antibodies (e.g., bispecific antibodies), monovalent antibodies, multivalent antibodies, antigen-binding antibody fragments (e.g., Fab′, F(ab′)2, Fab, Fv, rIgG, and scFv fragments), antibody fusions, and synthetic antibodies (or antibody mimetics).

“Full-length antibody,” “intact antibody,” or “whole antibody” are used herein interchangeably to refer to an antibody having a structure substantially similar to a native antibody structure or having heavy chains that contain an Fc region as defined herein.

“Antibody fragment” or “antigen binding fragment” refers to a portion of a full-length antibody which is capable of binding the same antigen as the full-length antibody. Examples of antibody fragments include, but are not limited to, Fv, Fab, Fab′, Fab′-SH, F(ab′)2; diabodies; linear antibodies; single-chain antibody molecules (e.g., scFv); and multispecific antibodies formed from antibody fragments.

“Class” of an antibody refers to the type of constant domain or constant region possessed by its heavy chain. There are five major classes of antibodies: IgA, IgD, IgE, IgG, and IgM, and several of these are further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

“Variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) (see, e.g., Kindt et al., Kuby Immunology, 6th ed., W.H. Freeman and Co., page 91). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (see, e.g., Portolano et al., J. Immunol. 150:880-887 (1993); Clarkson et al., Nature 352:624-628 (1991)).

“Complementarity determining region,” or “CDR,” as used herein, refers to the regions within the hypervariable regions of the variable domain which have the highest sequence variability and/or are involved in antigen recognition. Generally, native antibodies comprise four chains with six CDRs; three in the heavy chain variable domains, VH (H1, H2, H3), and three in the light chain variable domains, VL (L1, L2, L3). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35 of H1, 50-65 of H2, and 95-102 of H3. (Kabat et al., supra). With the exception of CDR-H1 in VH, CDRs generally comprise the amino acid residues that form the hypervariable loops.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

“Monoclonal antibody” as used herein refers to an antibody obtained from a substantially homogeneous population of antibodies, i.e., the individual antibodies comprising the population are identical and/or bind the same epitope, except for possible variant antibodies (e.g., variant antibodies contain mutations that occur naturally or arise during production of a monoclonal antibody, and generally are present in minor amounts). In contrast to polyclonal antibody preparations, which typically include different antibodies directed against different determinants (epitopes), each monoclonal antibody of a monoclonal antibody preparation is directed against a single determinant on an antigen. Thus, the term “monoclonal” indicates the character of the antibody as being obtained from a substantially homogeneous population of antibodies and is not to be construed as requiring production of the antibody by any particular method. For example, the monoclonal antibodies to be used may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Chimeric antibody” refers to an antibody in which a portion of the heavy and/or light chain is derived from a particular source or species, while the remainder of the heavy and/or light chain is derived from a different source or species.

“Humanized antibody” refers to a chimeric antibody comprising amino acid sequences from non-human HVRs and amino acid sequences from human FRs. In certain embodiments, a humanized antibody will comprise substantially all of at least one, and typically two, variable domains, in which all or substantially all of the FTVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

“Affinity” refers to the strength of the sum total of noncovalent interactions between a single binding site of a molecule (e.g., an antibody) and its binding partner (e.g., an antigen). “Binding affinity” refers to intrinsic binding affinity which reflects a 1:1 interaction between members of a binding pair (e.g., antibody and antigen). The affinity of a molecule X for its partner Y can generally be represented by the equilibrium dissociation constant (KD). Affinity can be measured by common methods known in the art, including those described herein. Specific illustrative and exemplary embodiments for measuring binding affinity are described in the following.

“Binds specifically” or “specific binding” refers to binding of an antibody to an antigen with an affinity value of no more than about 1×10⁻⁷ M.

“Treatment,” “treat” or “treating” refers to clinical intervention in an attempt to alter the natural course of a disorder in a subject being treated and can be performed either for prophylaxis or during the course of clinical pathology. Desired results of treatment can include, but are not limited to, preventing occurrence or recurrence of the disorder, alleviation of symptoms, diminishment of any direct or indirect pathological consequences of the disorder, preventing metastasis, decreasing the rate of progression, amelioration or palliation of a disease state, and remission or improved prognosis. For example, treatment of cancer or virus infection can include administration of a therapeutically effective amount of pharmaceutical composition comprising a PD-L1 antibody-IL-10 fusion protein to a subject to prevent, delay development of, slow progression of, or eradicate cancer or virus infection.

“Pharmaceutical composition” or “composition” or “formulation” refers to a preparation in a form that allows the biological activity of the active ingredient(s) to be effective, and which contain no additional components which are toxic to the subjects to which the formulation is administered.

“Sole active agent,” as used herein, refers an active agent in a pharmaceutical formulation that is the only active agent present in that formulation that provides, or would be expected to provide, the relevant pharmacological effect to treat the subject for the condition being treated. A pharmaceutical formulation comprising a sole active agent does not exclude the presence of one or more non-active agents, such as e.g., a pharmaceutically acceptable carrier, in the formulation. A “non-active agent” is an agent that would not be expected to provide, or otherwise significantly contribute to, the relevant pharmacological effect intended to treat the subject for the condition.

“Pharmaceutically acceptable carrier” refers to an ingredient in a pharmaceutical formulation, other than an active ingredient, which is nontoxic to the subject to whom it is administered. A pharmaceutically acceptable carrier includes, but is not limited to, a buffer, excipient, stabilizer, or preservative.

“Therapeutically effective amount,” as used herein, refers to the amount of an active ingredient or agent (e.g., a pharmaceutical composition) to achieve a desired therapeutic or prophylactic result, e.g., to treat or prevent a disease, disorder, or condition in a subject. In the case of a subject with cancer or virus infection, the therapeutically effective amount of the therapeutic agent is an amount that reduces, prevents, inhibits, and/or relieves to some extent one or more of the symptoms associated with the cancer or the virus infection, including the viral load, and/or the amount of viral Ag detectable in the subject. For therapeutic treatment of cancer or virus infection, efficacy in vivo can, for example, be measured by assessing the duration, severity, and/or recurrence of symptoms, the response rate (RR), duration of response, and/or quality of life.

“Subject” refers to a mammal, including but not limited to, primates (e.g., humans and non-human primates such as monkeys), rodents (e.g., mice and rats), rabbits, and domesticated animals (e.g., cows, sheep, cats, dogs, and horses).

“Subject in need” as referred to herein includes patients with an HBV infection, such as an HBV carrier, one with chronic HBV infection, one with HBV persistence, or one at risk of HBV infection.

“Immune checkpoint molecule,” as used herein, refers to a molecule that functions to regulate an immune system pathway and thereby prevent it from attacking cells unnecessarily. Many immune checkpoint molecules are targets for immunotherapy (e.g., with blocking antibodies) in the treatment of cancer and viral infections. Exemplary immune checkpoint molecules currently targeted for immunotherapy include PD1, PD-L1, CTLA-4, TIGIT, LAG3, PVRIG, KIR, TIM-3, CRTAM, BTLA, CD244, CD160, LIGHT, GITR, 4-1BB, OX40, CD27, TMIGD2, ICOS, CD40, CD47, SIRPa, NKG2D, NKG2A, TNFRSF25, CD33, CEA, Epcam, GPC3, CD200, CD200R, CD73, CD83, CD39, TRAIL, CD226, and VISTA.

### Detailed Description of Various Embodiments

### I. IL-10

The human IL-10 cytokine is a homodimeric protein of two 178 amino acid polypeptide subunits. IL-10 signals through a receptor complex consisting of two IL-10 receptor-1 (IL-10Ra subunit) and two IL-10 receptor-2 (IL-10RB subunit) proteins. Consequently, the functional receptor consists of four IL-10 receptor molecules. Binding of IL-10 to IL-10Ra induces STAT3 signaling via the phosphorylation of the cytoplasmic tails of IL-10 receptor by JAKI and Tyk2. IL-10 is primarily produced by monocytes and, to a lesser extent, lymphocytes, namely type-II T helper cells (T_{H}2), mast cells, CD4⁺CD25⁺Foxp3⁺ regulatory T cells, and in a certain subset of activated T cells and B cells. IL-10 can be produced by monocytes upon PD1 triggering. Table 1 below provides a summary description of the amino sequences of the human IL-10 polypeptide used in the Examples of the present disclosure, and their sequence identifiers. The sequences also are included in the accompanying Sequence Listing.

TABLE 1
Recombinant IL-10 polypeptides and polypeptide linkers

| | | SEQ ID |
|---|---|---|
| Description | Sequence | NO: |
| IL-10 | SPGQGTQSENSCTHFPGNLPNMLRDLRDAFSRVKTFFQMKDQLDN | 73 |
| | LLLKESLLEDFKGYLGCQALSEMIQFYLEEVMPQAENQDPDIKAH | |
| | NVSLGENLKTLRLRLRRCHRFLPCENKSKAVEQVKNAFNKLQEKG | |
| | IYKAMSEFDIFINYIEAYMTMKIRN | |
| IL-10 | SPGQGTQSENSCTHFPGNLPNMLRDLRDAFSRVKTFFQMKDQLDN | 172 |
| R104Q | LLLKESLLEDFKGYLGCQALSEMIQFYLEEVMPQAENQDPDIKAH | |
| | VNSLGENLKTLRLQLRRCHRFLPCENKSKAVEQVKNAFNKLQEKG | |
| | IYKAMSEFDIFINYIEAYMTMKIRN | |
| IL-10 | SPGQGTQSENSCTHFPGNLPNMLRDLRDAFSRVKTFFQMKDQLDN | 173 |
| R107A | LLLKESLLEDFKGYLGCQALSEMIQFYLEEVMPQAENQDPDIKAH | |
| | VNSLGENLKTLRLRLRACHRFLPCENKSKAVEQVKNAFNKLQEKG | |
| | IYKAMSEFDIFINYIEAYMTMKIRN_ | |
| IL-10 | SPGQGTQSENSCTHFPGNLPNMLRDLRDAFSRVKTFFQMKDQLDN | 174 |
| R107E | LLLKESLLEDFKGYLGCQALSEMIQFYLEEVMPQAENQDPDIKAH | |
| | VNSLGENLKTLRLRLRECHRFLPCENKSKAVEQVKNAFNKLQEKG | |
| | IYKAMSEFDIFINYIEAYMTMKIRN | |
| IL-10 | SPGQGTQSENSCTHFPGNLPNMLRDLRDAFSRVKTFFQMKDQLDN | 175 |
| R107Q | LLLKESLLEDFKGYLGCQALSEMIQFYLEEVMPQAENQDPDIKAH | |
| | VNSLGENLKTLRLRLRQCHRFLPCENKSKAVEQVKNAFNKLQEKG | |
| | IYKAMSEFDIFINYIEAYMTMKIRN | |
| IL-10 | SPGQGTQSENSCTHFPGNLPNMLRDLRDAFSRVKTFFQMKDQLDN | 176 |
| R107D | LLLKESLLEDFKGYLGCQALSEMIQFYLEEVMPQAENQDPDIKAH | |
| | VNSLGENLKTLRLRLRDCHRFLPCENKSKAVEQVKNAFNKLQEKG | |
| | IYKAMSEFDIFINYIEAYMTMKIRN_ | |
| IL-10 | SPGQGTQSENSCTHFPGNLPNMLRDLRDAFSRVKTFFQMKDQLDN | 177 |
| R104Q/ | LLLKESLLEDFKGYLGCQALSEMIQFYLEEVMPQAENQDPDIKAH | |
| R107A | VNSLGENLKTLRLQLRACHRFLPCENKSKAVEQVKNAFNKLQEKG | |
| (QA) | IYKAMSEFDIFINYIEAYMTMKIRN | |
| IL-10 | SPGQGTQSENSCTHFPGNLPNMLRDLRDAFSRVKTFFQMKDQLDN | 178 |
| R104Q/ | LLLKESLLEDFKGYLGCQALSEMIQFYLEEVMPQAENQDPDIKAH | |
| R107E | VNSLGENLKTLRLQLRECHRFLPCENKSKAVEQVKNAFNKLQEKG | |
| | IYKAMSEFDIFINYIEAYMTMKIRN | |
| IL-10 | SPGQGTQSENSCTHFPGNLPNMLRDLRDAFSRVKTFFQMKDQLDN | 179 |
| R104Q/ | LLLKESLLEDFKGYLGCQALSEMIQFYLEEVMPQAENQDPDIKAH | |
| R107Q | VNSLGENLKTLRLQLRQCHRFLPCENKSKAVEQVKNAFNKLQEKG | |
| | IYKAMSEFDIFINYIEAYMTMKIRN | |
| IL-10 | SPGQGTQSENSCTHFPGNLPNMLRDLRDAFSRVKTFFQMKDQLDN | 180 |
| R104Q/ | LLLKESLLEDFKGYLGCQALSEMIQFYLEEVMPQAENQDPDIKAH | |
| R107D | VNSLGENLKTLRLQLRDCHRFLPCENKSKAVEQVKNAFNKLQEKG | |
| | IYKAMSEFDIFINYIEAYMTMKIRN | |
| Linker | LGGGGSGGGGSGGGG | 74 |
| Linker | GGGGSGGGGSGGGG | 75 |
| Linker | GGGGSGGGGSGGGGS | 76 |
| Linker | GGGGSGGGGSGGGGSGGGGS | 77 |
| Linker | GGGGSGGGGSGGGGSGGGGSGGGGS | 78 |
| Linker | GGGGSGGGGSGGGGSGGGGSGGGGSGGGGS | 79 |

In addition to the naturally-occurring human IL-10, a variety of engineered and/or synthetically modified IL-10 polypeptides that retain the cytokine functions of IL-10 are known in the art. The PEGylated IL-10, Pegilodecakin, has been shown to retain the anti-tumor immune surveillance function of naturally-occurring human IL-10. See, Naing, A. et al. “PEGylated IL-10 (Pegilodecakin) Induces Systemic Immune Activation, CD8+ T Cell Invigoration and Polyclonal T Cell Expansion in Cancer Patients.” Cancer Cell 34, 775-791. (2018). The engineered IL-10 variant R5A11 has been shown to have higher affinity to IL10R2, exhibit enhanced signaling activities in human CD8+ T cells, and enhances the anti-tumor function of CAR-T cells. See, Gorby, C. et al. “Engineered IL-10 variants elicit potent immunomodulatory effects at low ligand doses.” Sci Signal 13, (2020). The IL-10 from Epstein-Barr virus has weaker binding to the IL-10R1, but retains the immunosuppressive cytokine activities of human IL10, while having lost the ability to induce immunostimulatory activities with some cells. See, Yoon, S. I. et al. “Epstein-Barr virus IL-10 engages IL-10R1 by a two-step mechanism leading to altered signaling properties.” J Biol Chem 287, 26586-26595 (2012). U.S. Pat. No. 7,749,490 B2 and US 2017/0015747 A1 described engineered IL-10 mutants (e.g., F129S-IL10) that exhibit less immunostimulatory activity in MC/9 cell proliferation assay. Generally, it is contemplated that any engineered or modified version of IL-10 polypeptide that retains some IL-10 cytokine function can be used in any of the anti-PD-L1/IL10 fusion protein compositions and methods of the present disclosure.

In some embodiments, the IL-10 may be an IL-10 mutein comprising one or more substitution on amino acids in position 104, position 107, and a combination thereof, relative to amino acids of wild-type IL-10. In one embodiment, the substitution may comprise: R104Q; any one of R107A, R107E, R107Q and R107D; or a combination thereof. In some embodiment, the IL-10 mutein of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 73, 172, 173, 174, 175, 176, 177, 178, 179 or 180, and further include the amino acid substitutions corresponding to the following amino acid substitutions: (a) R104Q; (b) R107A; (c) R107E; (d) R107Q; (e) R107D; (f) R104Q/R107A; (g) R104Q/R107E; (h) R104Q/R107Q; and (i) R104Q/R107D.

### III. Anti-PD-L1 Antibodies and Anti-PD-L1/IL-10 Fusion Proteins

In some embodiments, the present disclosure provides structures of anti-PD-L1 antibodies in terms of the amino acid and encoding nucleotide sequences of the various well-known immunoglobulin features (e.g., CDRs, FRs, V_{H}, V_{L} domains, and full-length heavy and light chains). Table 2 below provides a summary description of anti-PD-L1 antibody sequences of the present disclosure, including antibody fusions, and their sequence identifiers. The sequences are included in the accompanying Sequence Listing.

TABLE 2
Anti-PD-L1 antibody fusion sequences

| | | | SEQ ID |
|---|---|---|---|
| Protein Name | Description | Sequence | NO: |
| Atezolizumab/ | Heavy Chain | EVQLVESGGGLVQPGGSLRLSCAASGFTFSDSWIHWVRQ | 80 |
| IL10 | | APGKGLEWVAWISPYGGSTYYADSVKGRFTISADTSKNT | |
| | | AYLQMNSLRAEDTAVYYCARRHWPGGFDYWGQGTLVT | |
| | | VSSastkgpsvfplapsskstsggtaalgclvkdyfpepvtvswnsgaltsgvhtfpavl | |
| | | qssglyslssvvtvpssslgtqtyicnvnhkpsntkvdkkvepkscdkthtcppcpap | |
| | | ellggpsvflfppkpkdtlmisrtpevtcvvvdvshedpevkfnwyvdgvevhn | |
| | | aktkpreeqyastyrvvsvltvlhqdwlngkeykckvsnkalpapiektiskakg | |
| | | qprepqvytlppsreemtknqvsltclvkgfypsdiavewesngqpennykttp | |
| | | pvldsdgsfflyskltvdksrwqqgnvfscsvmhealhnhytqkslslspgkLG | |
| | | GGGSGGGGSGGGGSPGQGTQSENSCTHFPGNLPNMLRDL | |
| | | RDAFSRVKTFFQMKDQLDNLLLKESLLEDFKGYLGCQAL | |
| | | SEMIQFYLEEVMPQAENQDPDIKAHVNSLGENLKTLRLRL | |
| | | RRCHRFLPCENKSKAVEQVKNAFNKLQEKGIYKAMSEFD | |
| | | IFINYIEAYMTMKIRN | |
| | Light Chain | DIQMTQSPSSLSASVGDRVTITCRASQDVSTAVAWQQKP | 143 |
| | | GKAPKLLIYSASFLYSGVPSRFSGSGSGTDFTLTISSLQPED | |
| | | FATYYCQQYLYHPATFGQGTKVEIKRrtvaapsvfifppsdeqlksg | |
| | | tasvvcllnnfypreakvqwkvdnalqsgnsqesvteqdskdstyslsstltlskadyek | |
| | | hkvyacevthqglsspvtksfnrgec | |
| Avelumab/IL10 | Heavy Chain | EVQLLESGGGLVQPGGSLRLSCAASGFTFSSYIMMWVRQ | 81 |
| | | APGKGLEWVSSIYPSGGITFYADTVKGRFTISRDNSKNTL | |
| | | YLQMNSLRAEDTAVYYCARIKLGTVTTVDYWGQGTLVT | |
| | | VSSastkgpsvfplapsskstsggtaalgclvkdyfpepvtvswnsgaltsgvhtfpavl | |
| | | qssglyslssvvtvpssslgtqtyicnvnhkpsntkvdkkvepkscdkthtcppcpap | |
| | | ellggpsvflfppkpkdtlmisrtpevtcvvvdvshedpevkfnwyvdgvevhna | |
| | | ktkpreeqynstyrvvsvltvlhqdwlngkeykckvsnkalpapiektiskakgq | |
| | | prepqvytlppsreemtknqvsltclvkgfypsdiavewesngqpennykttpp | |
| | | vldsdgsfflyskltvdksrwqqgnvfscsvmhealhnhytqkslslspgkLGG | |
| | | GGSGGGGSGGGGSPGQGTQSENSCTHFPGNLPNMLRDLR | |
| | | DAFSRVKTFFQMKDQLDNLLLKESLLEDFKGYLGCQALS | |
| | | EMIQFYLEEVMPQAENQDPDIKAHVNSLGENLKTLRLRL | |
| | | RRCHRFLPCENKSKAVEQVKNAFNKLQEKGIYKAMSEFD | |
| | | IFINYIEAYMTMKIRN | |
| | Light Chain | QSALTQPASVSGSPGQSITISCTGTSSDVGGYNYVSWYQQ | 145 |
| | | HPGKAPKLMIYDVSNRPSGVSNRFSGSKSGNTASLTISGL | |
| | | QAEDEADYYCSSYTSSSTRVFGTGTKVTVLgqpkanptvtlfpp | |
| | | sseelqankatlvclisdfypgavtvawkadgspvkagvettkpskqsnnkyaassylsl | |
| | | tpeqwkshrsyscqvthegstvektvaptecs | |
| Durvalumab/ | Heavy Chain | EVQLVESGGGLVQPGGSLRLSCAASGFTFSRYWMSWVR | 82 |
| IL10 | | QAPGKGLEWVANIKQDGSEKYYVDSVKGRFTISRDNAK | |
| | | NSLYLQMNSLRAEDTAVYYCAREGGWFGELAFDYWGQ | |
| | | GTLVTVSSastkgpsvfplapsskstsggtaalgclvkdyfppvtvswnsgaltsgv | |
| | | htfpavlqssglyslssvvtvpssslgtqtyicnvnhkpsntkvdkkvepkscdkthtc | |
| | | ppcpapellggpsvflfppkpkdtlmisrtpevtcvvvdvshedpevkfnwyvd | |
| | | gvevhnaktkpreeqyastyrvvsvltvlhqdwlngkeykckvsnkalpapiek | |
| | | tiskakgqprepqvytlppsreemtknqvsltclvkgfypsdiavewesngqpe | |
| | | nnykttppvldsdgsfflyskltvdksrwqqgnvfscsvmhealhnhytqkslsls | |
| | | pgkLGGGGSGGGGSGGGGSPGQGTQSENSCTHFPGNLPN | |
| | | MLRDLRDAFSRVKTFFQMKDQLDNLLLKESLLEDFKGYL | |
| | | GCQALSEMIQFYLEEVMPQAENQDPDIKAHVNSLGENLK | |
| | | TLRLRLRRCHRFLPCENKSKAVEQVKNAFNKLQEKGIYK | |
| | | AMSEFDIFINYIEAYMTMKIRN | |
| | Light Chain | EIVLTQSPGTLSLSPGERATLSCRASQRVSSSYLAWYQQK | 147 |
| | | PGQAPRLLIYDASSRATGIPDRESGSGSGTDFTLTISRLEPE | |
| | | DFAWYYCQQYGSLPWTFGQGTKVEIKrtvaapsvfifppsdeqlks | |
| | | gtasvvcllnnfypreakvqwkvdnalqsgnsqesvteqdskdstyslsstltlskadye | |
| | | khkvyacevthqglsspvtksfnrgec | |
| PHS102/IL10 | Heavy Chain | EVQLVESGGGLVQPGGSLRLSCAASGFTITDSFIHWVRQA | 83 |
| | | PGKGLEWVARIDPYGGTTNYADSVKGRFTISADTSKNTA | |
| | | YLQMNSLRAEDTAVYYCARAYSWFSDYWGQGTLVTVSS | |
| | | astkgpsvfplapsskstsggtaalgclvkdyfpepvtvswnsgaltsgvhtfpavlqss | |
| | | glyslssvvtvpssslgtqtyicnvnhkpsntkvdkkvepkscdkthtcppcpapellgg | |
| | | psvflfppkpkdtlmisrtpevtcvvvdvshedpevkfnwyvdgvevhnaktk | |
| | | preeqyastyrvvsvltvlhqdwlngkeykckvsnkalpapiektiskakgqpre | |
| | | pqvytlppsreemtknqvsltclvkgfypsdiavewesngqpennykttppvlds | |
| | | dgsfflyskltvdksrwqqgnvfscsvmhealhnhytqkslslspgkLGGGGS | |
| | | GGGGSGGGGSPGQGTQSENSCTHFPGNLPNMLRDLRDAF | |
| | | SRVKTFFQMKDQLDNLLLKESLLEDFKGYLGCQALSEMI | |
| | | QFYLEEVMPQAENQDPDIKAHVNSLGENLKTLRLRLRRC | |
| | | HRFLPCENKSKAVEQVKNAFNKLQEKGIYKAMSEFDIFIN | |
| | | YIEAYMTMKIRN | |
| | Light Chain | DIQMTQSPSSLSASVGDRVTITCRAGQDVYKAVAWYQQ | 141 |
| | | KPGKAPKLLIYWSTNLYSGVPSRFSGSGSGTDFTLTISSLQ | |
| | | PEDFATYYCQQGSTWPLTFGQGTKVEIKrtvaapsvfifppsdeql | |
| | | ksgtasvvcllnnfypreakvqwkvdnalqsgnsqesvteqdskdstyslsstltlskad | |
| | | yekhkvyacevthqglsspvtksfnrgec | |
| HSYPP31F/IL10 | Heavy Chain | EVQLVESGGGLVQPGGSLRLSCAASGFTIEDSYIHWVRQ | 132 |
| | | APGKGLEWVARIDPITGLTHYADSVKGRFTISADTSKNTA | |
| | | YLQMNSLRAEDTAVYYCARAFSWFPDYWGQGTLVTVSS | |
| | | astkgpsvfplapsskstsggtaalgclvkdyfpepvtvswnsgaltsgvhtfpavlqss | |
| | | glyslssvvtvpssslgtqtyicnvnhkpsntkvdkkvepkscdkthtcppcpapell | |
| | | ggpsvflfppkpkdtlmisrtpevtcvvvdvshedpevkfnwyvdgvevhnak | |
| | | tkpreeqyastyrvvsvltvlhqdwlngkeykckvsnkalpapiektiskakgqp | |
| | | repqvytlppsreemtknqvsltclvkgfypsdiavewesngqpennykttppv | |
| | | ldsdgsfflyskltvdksrwqqgnvfscsvmhealhnhytqkslslspgkLGGG | |
| | | GSGGGGSGGGGSPGQGTQSENSCTHFPGNLPNMLRDLRD | |
| | | AFSRVKTFFQMKDQLDNLLLKESLLEDFKGYLGCQALSE | |
| | | MIQFYLEEVMPQAENQDPDIKAHVNSLGENLKTLRLRLR | |
| | | RCHRFLPCENKSKAVEQVKNAFNKLQEKGIYKAMSEFDI | |
| | | FINYIEAYMTMKIRN | |
| | Light Chain | DIQMTQSPSSLSASVGDRVTITCRAGQDVYKAVAWYQQ | 133 |
| | | KPGKAPKLLIYWSTNLYSGVPSRFSGSGSGTDFTLTISSLQ | |
| | | PEDFATYYCQQIMEPPVTFGQGTKVEIKrtvaapsvfifppsdeql | |
| | | ksgtasvvcllnnfypreakvqwkvdnalqsgnsqesvteqdskdstyslsstltlskad | |
| | | yekhkvyacevthqglsspvtksfnrgec | |
| HSYPP411C/ | Heavy Chain | EVQLVESGGGLVQPGGSLRLSCAASGFTIDNTYIHWVRQ | 134 |
| IL10 | | APGKGLEWVARIDPANGKTTYADSVKGRFTISADTSKNT | |
| | | ANLQMNSLRAEDTAVYYCARSFSWWADYWGQGTLVTV | |
| | | SSastkgpsvfplapsskstsggtaalgclvkdyfpepvtvswnsgaltsgvhtfpavlq | |
| | | ssglyslssvvtvpssslgtqtyicnvnhkpsntkvdkkvepkscdkthtcppcpape | |
| | | llggpsvflfppkpkdtlmisrtpevtcvvvdvshedpevkfnwyvdgvevhna | |
| | | ktkpreeqyastyrvvsvltvlhqdwlngkeykckvsnkalpapiektiskakgq | |
| | | prepqvytlppsreemtknqvsltclvkgfypsdiavewesngqpennykttpp | |
| | | vldsdgsfflyskltvdksrwqqgnvfscsvmhealhnhytqkslslspgkLGG | |
| | | GGSGGGGSGGGGSPGQGTQSENSCTHFPGNLPNMLRDLR | |
| | | DAFSRVKTFFQMKDQLDNLLLKESLLEDFKGYLGCQALS | |
| | | EMIQFYLEEVMPQAENQDPDIKAHVNSLGENLKTLRLRL | |
| | | RRCHRFLPCENKSKAVEQVKNAFNKLQEK | |
| | Light Chain | DIQMTQSPSSLSASVGDRVTITCRAGQDVYKAVAWYQQ | 135 |
| | | KPGKAPKLLIYWSTNLYSGVPSRFSGSGSGTDFTLTISSLQ | |
| | | PEDFATYYCQQFNLQPTTFGQGTKVEIKrtvaapsvfifppsdeql | |
| | | ksgtasvvcllnnfypreakvqwkvdnalqsgnsqesvteqdskdstyslsstltlskad | |
| | | yekhkvyacevthqglsspvtksfnrgec | |
| YP7G/IL10 | Heavy Chain | EVQLVESGGGLVQPGGSLRLSCAASGFTIDDTYIHWVRQ | 140 |
| | | APGKGLEWVARIDPANGMTRYADSVKGRFMISADTSKN | |
| | | TAYLQMNSLRAEDTAVYYCARAFSWFPDYWGQGTLVT | |
| | | VSSastkgpsvfplapsskstsggtaalgclvkdyfpepvtvswnsgaltsgvhtfpav | |
| | | lqssglyslssvvtvpssslgtqtyicnvnhkpsntkvdkkvepkscdkthtcppcpa | |
| | | pellggpsvflfppkpkdtlmisrtpevtcvvvdvshedpevkfnwyvdgvevh | |
| | | naktkpreeqyastyrvvsvltvlhqdwlngkeykckvsnkalpapiektiskak | |
| | | gqprepqvytlppsreemtknqvsltclvkgfypsdiavewesngqpennyktt | |
| | | ppvldsdgsfflyskltvdksrwqqgnvfscsvmhealhnhytqkslslspgkL | |
| | | GGGGSGGGGSGGGGSPGQGTQSENSCTHFPGNLPNMLR | |
| | | DLRDAFSRVKTFFQMKDQLDNLLLKESLLEDFKGYLGCQ | |
| | | ALSEMIQFYLEEVMPQAENQDPDIKAHVNSLGENLKTLR | |
| | | LRLRRCHRFLPCENKSKAVEQVKNAFNKLQEKGIYKAM | |
| | | SEFDIFINYIEAYMTMKIRN | |
| | Light Chain | DIQMTQSPSSLSASVGDRVTITCRAGQDVYKAVAWYQQ | 141 |
| | | KPGKAPKLLIYWSTNLYSGVPSRFSGSGSGTDFTLTISSLQ | |
| | | PEDFATYYCQQGSTWPLTFGQGTKVEIKrtvaapsvfifppsdeql | |
| | | ksgtasvvcllnnfypreakvqwkvdnalqsgnsqesvteqdskdstyslsstltlskad | |
| | | yekhkvyacevthqglsspvtksfnrgec | |
| YP11F/IL10 | Heavy Chain | EVQLVESGGGLVQPGGSLRLSCAASGFTIDNTYIHWVRQ | 128 |
| | | APGKGLEWVARIDPVSGRTRYADSVKGRFTISADTSKNT | |
| | | ADLQMNSLRAEDTAVYYCARAFSWFPDYWGQGTLVTV | |
| | | SSastkgpsvfplapsskstsggtaalgclvkdyfpepvtvswnsgaltsgvhtfpavlq | |
| | | ssglyslssvvtvpssslgtqtyicnvnhkpsntkvdkkvepkscdkthteppcpape | |
| | | llggpsvflfppkpkdtlmisrtpevtcvvvdvshedpevkfnwyvdgvevhna | |
| | | ktkpreeqyastyrvvsvltvlhqdwlngkeykckvsnkalpapiektiskakgq | |
| | | prepqvytlppsreemtknqvsltclvkgfypsdiavewesngqpennykttpp | |
| | | vldsdgsfflyskltvdksrwqqgnvfscsvmhealhnhytqkslslspgkLGG | |
| | | GGSGGGGSGGGGSPGQGTQSENSCTHFPGNLPNMLRDLR | |
| | | DAFSRVKTFFQMKDQLDNLLLKESLLEDFKGYLGCQALS | |
| | | EMIQFYLEEVMPQAENQDPDIKAHVNSLGENLKTLRLRL | |
| | | RRCHRFLPCENKSKAVEQVKNAFNKLQEKGIYKAMSEF | |
| | | DIFINYIEAYMTMKIRN | |
| | Light Chain | DIQMTQSPSSLSASVGDRVTITCRAGQDVYKAVAWYQQ | 129 |
| | | KPGKAPKLLIYWSTNLYSGVPSRFSGSGSGTDFTLTISSLQ | |
| | | PEDFATYYCQQFGATPITFGQGTKVEIKrtvaapsvfifppsdeqlk | |
| | | sgtasvvcllnnfypreakvqwkvdnalqsgnsqesvteqdskdstyslsstltlskady | |
| | | ekhkvyacevthqglsspvtksfnrgec | |
| YT6D/IL10 | Heavy Chain | EVQLVESGGGLVQPGGSLRLSCAASGFTISDGYIHWVRQ | 126 |
| | | APGKGLEWVARIDPLTGRTMYADSVKGRFTISADTSKNT | |
| | | AYLQMNSLRAEDTAVYYCARAFSWWPDYWGQGTLVTV | |
| | | SSastkgpsvfplapsskstsggtaalgclvkdyfpepvtvswnsgaltsgvhtfpavlq | |
| | | ssglyslssvvtvpssslgtqtyicnvnhkpsntkvdkkvepkscdkthteppcpape | |
| | | llggpsvflfppkpkdtlmisrtpevtcvvvdvshedpevkfnwyvdgvevhna | |
| | | ktkpreeqyastyrvvsvltvlhqdwlngkeykckvsnkalpapiektiskakgq | |
| | | prepqvytlppsreemtknqvsltclvkgfypsdiavewesngqpennykttpp | |
| | | vldsdgsfflyskltvdksrwqqgnvfscsvmhealhnhytqkslslspgkLGG | |
| | | GGSGGGGSGGGGSPGQGTQSENSCTHFPGNLPNMLRDLR | |
| | | DAFSRVKTFFQMKDQLDNLLLKESLLEDFKGYLGCQALS | |
| | | EMIQFYLEEVMPQAENQDPDIKAHVNSLGENLKTLRLRL | |
| | | RRCHRFLPCENKSKAVEQVKNAFNKLQEKGIYKAMSEF | |
| | | DIFINYIEAYMTMKIRN | |
| | Light Chain | DIQMTQSPSSLSASVGDRVTITCRAGQDVYKAVAWYQQ | 127 |
| | | KPGKAPKLLIYWSTNLYSGVPSRFSGSGSGTDFTLTISSLQ | |
| | | PEDFATYYCQQIMSPPPTFGQGTKVEIKrtvaapsvfifppsdeqlk | |
| | | sgtasvvcllnnfypreakvqwkvdnalqsgnsqesvteqdskdstyslsstltlskady | |
| | | ekhkvyacevthqglsspvtksfnrgec | |
| YT7A/IL10 | Heavy Chain | EVQLVESGGGLVQPGGSLRLSCAASGFTIDNTYIHWVRQ | 136 |
| | | APGKRLEWVARIDPANGVTRYADSVKGRFTISADTSKNT | |
| | | AYLQMNSLRAEDTAVYYCARAFSWFPDYWGQGTLVTV | |
| | | SSastkgpsvfplapsskstsggtaalgclvkdyfpepvtvswnsgaltsgvhtfpavlq | |
| | | ssglyslssvvtvpssslgtqtyicnvnhkpsntkvdkkvepkscdkthtcppcpape | |
| | | llggpsvflfppkpkdtlmisrtpevtcvvvdvshedpevkfnwyvdgvevhna | |
| | | ktkpreeqyastyrvvsvltvlhqdwlngkeykckvsnkalpapiektiskakgq | |
| | | prepqvytlppsreemtknqvsltclvkgfypsdiavewesngqpennykttpp | |
| | | vldsdgsfflyskltvdksrwqqgnvfscsvmhealhnhytqkslslspgkLGG | |
| | | GGSGGGGSGGGGSPGQGTQSENSCTHFPGNLPNMLRDLR | |
| | | DAFSRVKTFFQMKDQLDNLLLKESLLEDFKGYLGCQALS | |
| | | EMIQFYLEEVMPQAENQDPDIKAHVNSLGENLKTLRLRL | |
| | | RRCHRFLPCENKSKAVEQVKNAFNKLQEKGIYKAMSEF | |
| | | DIFINYIEAYMTMKIRN | |
| | Light Chain | DIQMTQSPSSLSASVGDRVTITCRAGQDVYKAVAWYQQ | 137 |
| | | KPGKAPKLLIYWSTNLYSGVPSRFSGSGSGTDFTLTISSLQ | |
| | | PEDFATYYCQQYGIAPPTFGQGTKVEIKrtvaapsvfifppsdeqlk | |
| | | sgtasvvcllnnfypreakvqwkvdnalqsgnsqesvteqdskdstyslsstltlskady | |
| | | ekhkvyacevthqglsspvtksfnrgec | |
| YT7H/IL10 | Heavy Chain | EVQLVESGGGLVQPGGSLRLSCAASGFTITDGYIHWVRQ | 138 |
| | | APGKGLEWVARIDPYNGRTNYADSVKGRFTISADTSKNT | |
| | | AYLQMNSLRAEDTAVYYCARAFSWFPDYWGQGTLVTV | |
| | | SSastkgpsvfplapsskstsggtaalgclvkdyfpepvtvswnsgaltsgvhtfpavlq | |
| | | ssglyslssvvtvpssslgtqtyicnvnhkpsntkvdkkvepkscdkthtcppcpape | |
| | | llggpsvflfppkpkdtlmisrtpevtcvvvdvshedpevkfnwyvdgvevhna | |
| | | ktkpreeqyastyrvvsvltvlhqdwlngkeykckvsnkalpapiektiskakgq | |
| | | prepqvytlppsreemtknqvsltclvkgfypsdiavewesngqpennykttpp | |
| | | vldsdgsfflyskltvdksrwqqgnvfscsvmhealhnhytqkslslspgkLGG | |
| | | GGSGGGGSGGGGSPGQGTQSENSCTHFPGNLPNMLRDLR | |
| | | DAFSRVKTFFQMKDQLDNLLLKESLLEDFKGYLGCQALS | |
| | | EMIQFYLEEVMPQAENQDPDIKAHVNSLGENLKTLRLRL | |
| | | RRCHRFLPCENKSKAVEQVKNAFNKLQEKGIYKAMSEF | |
| | | DIFINYIEAYMTMKIRN | |
| | Light Chain | DIQMTQSPSSLSASVGDRVTITCRAGQDVYKAVAWYQQ | 139 |
| | | KPGKAPKLLIYWSTNLYSGVPSRFSGSGSGTDFTLTISSLQ | |
| | | PEDFATYYCQQHGNAPITFGQGTKVEIKrtvaapsvfifppsdeql | |
| | | ksgtasvvcllnnfypreakvqwkvdnalqsgnsqesvteqdskdstyslsstltlskad | |
| | | yekhkvyacevthqglsspvtksfnrgec | |
| YT10H/IL10 | Heavy Chain | EVQLVESGGGLVQPGGSLRLSCAASGFTISDAYIHWVRQ | 130 |
| | | APGKGLEWVARIEPLSGRTDYADSVKGRFTISADTSKNT | |
| | | AYLQMNSLRAEDTAVYYCARAFSWFMDYWGQGTLVTV | |
| | | SSastkgpsvfplapsskstsggtaalgclvkdyfpepvtvswnsgaltsgvhtfpavlq | |
| | | ssglyslssvvtvpssslgtqtyicnvnhkpsntkvdkkvepkscdkthtcppcpape | |
| | | llggpsvflfppkpkdtlmisrtpevtcvvvdvshedpevkfnwyvdgvevhna | |
| | | ktkpreeqyastyrvvsvltvlhqdwlngkeykckvsnkalpapiektiskakgq | |
| | | prepqvytlppsreemtknqvsltclvkgfypsdiavewesngqpennykttpp | |
| | | vldsdgsfflyskltvdksrwqqgnvfscsvmhealhnhytqkslslspgkLGG | |
| | | GGSGGGGSGGGGSPGQGTQSENSCTHFPGNLPNMLRDLR | |
| | | DAFSRVKTFFQMKDQLDNLLLKESLLEDFKGYLGCQALS | |
| | | EMIQFYLEEVMPQAENQDPDIKAHVNSLGENLKTLRLRL | |
| | | RRCHRFLPCENKSKAVEQVKNAFNKLQEKGIYKAMSEF | |
| | | DIFINYIEAYMTMKIRN | |
| | Light Chain | DIQMTQSPSSLSASVGDRVTITCRAGQDVYKAVAWYQQ | 131 |
| | | KPGKAPKLLIYWSTNLYSGVPSRFSGSGSGTDFTLMISSL | |
| | | QPEDFATYYCQQHDKTPTTFGQGTKVEIKrtvaapsvfifppsde | |
| | | qlksgtasvvcllnnfypreakvqwkvdnalqsgnsqesvteqdskdstyslsstltlsk | |
| | | adyekhkvyacevthqglsspvtksfnrgec | |

Variable domain: UPPERCASE
Constant domain: lower case
Linker: UPPERCASE ITALICS
IL-10: UPPERCASE UNDERLINED
human IgG1 Fc fragment: Bold lower case

### Pharmaceutical Compositions and Formulations of Anti-PD-L1 Antibodies

The present disclosure also provides pharmaceutical compositions and pharmaceutical formulations comprising an anti-PD-L1 antibody-IL-10 fusion protein. In some embodiments, the present disclosure provides a pharmaceutical formulation comprising an anti-PD-L1 antibody-IL-10 fusion protein as described herein and a pharmaceutically acceptable carrier. In some embodiments, the anti-PD-L1 antibody-IL-10 fusion protein is the sole active agent of the pharmaceutical composition. Such pharmaceutical formulations can be prepared by mixing an anti-PD-L1 antibody-IL-10 fusion protein, having the desired degree of purity, with one or more pharmaceutically acceptable carriers. Typically, such antibody formulations can be prepared as an aqueous solution (see e.g., U.S. Pat. No. 6,171,586, and WO2006/044908) or as a lyophilized formulation (see e.g., U.S. Pat. No. 6,267,958).

Pharmaceutically acceptable carriers are generally nontoxic to recipients at the dosages and concentrations employed. A wide range of such pharmaceutically acceptable carriers are well-known in the art (see e.g., Remington's Pharmaceutical Sciences 16th edition, Osol, A. Ed. (1980)). Exemplary pharmaceutically acceptable carriers useful in the formulations of the present disclosure can include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Pharmaceutically acceptable carriers useful in the formulations of the present disclosure can also include interstitial drug dispersion agents, such as soluble neutral-active hyaluronidase glycoproteins (sHASEGP) (see e.g., US Pat. Publ. Nos. 2005/0260186 and 2006/0104968), such as human soluble PH-20 hyaluronidase glycoproteins (e.g., rHuPH20 or HYLENEX®, Baxter International, Inc.).

It is also contemplated that the formulations disclosed herein may contain active ingredients in addition to the anti-PD-L1-IL-10 fusion protein, as necessary for the particular indication being treated in the subject to whom the formulation is administered. Preferably, any additional active ingredient has activity complementary to that of the anti-PD-L1 antibody-IL-10 fusion protein activity and the activities do not adversely affect each other.

As disclosed elsewhere herein, including the Examples, it has been shown that the anti-PD-L1 antibodies of the present disclosure can be used in combination with an IL10 polypeptide to provide improved therapeutic effect in treating cancers. Accordingly, in some embodiments the present disclosure provides a pharmaceutical composition or formulation useful for treating a cancer comprising a PD-L1 antagonist (such as an anti-PD-L1) and an IL10 agonist (such as an IL10). In addition, to the use of the anti-PD-L1 antibodies of the present disclosure as PD-L1 antagonist in such a pharmaceutical formulation or composition, it is also contemplated that other antagonists can be used, including but not limited to a shRNA, a siRNA, a miRNA, a small molecule inhibitor of PD-L1, or a combination thereof. Small molecule inhibitors of PD-L1 useful in such pharmaceutical compositions or formulations can include known compounds in clinical development including, but not limited to, AUNP12 (Aurigene), CA-170 (Aurigene/Curis), and BMS-986189 (Bristol-Myers Squibb). Besides the anti-PD-L1 antibodies of the present disclosure, other known anti-PD-L1 antibodies useful in such a combination pharmaceutical composition or formulation with an IL10 can include any known antibodies that bind PD-L1, including those in clinical development for cancer treatment, such as Atezolizumab, Avelumab, Durvalumab, Lodapolimab, FAZ053 (BAP058-hum13), and MDX-1105, which are described elsewhere herein.

As described elsewhere herein, in some embodiments the present disclosure provides pharmaceutical composition or formulation for use in a combination therapy comprising a PD-L1 antagonist and an IL10 agonist. In some embodiments, this combination can be provided as a single pharmaceutical composition or formulation comprising an anti-PD-L1 antibody fusion having an anti-PD-L1 antibody covalently fused to an IL10 through a polypeptide linker, such as linker sequence of SEQ ID NO: 74, 75, 76, 77, 78, or 79. Examples demonstrating such anti-PD-L1 antibody fusions (e.g., PHS102/IL10) and their use in pharmaceutical compositions for reducing tumor volume in a range of syngeneic mouse cancer models is provided elsewhere herein including the Examples. Details about the generation, affinity maturation and characterization of anti-PD-L1 antibody fusion can be found in the examples appended to PCT publication no. WO2021231741A2, which is incorporated herein by reference.

In some embodiments, the pharmaceutical composition comprises the anti-PD-L1 antibody and an additional active agent for cancer treatment such as an immune checkpoint inhibitor. Checkpoint inhibitors useful in such embodiments include, but are not limited to, a second antibody comprising a specificity for an antigen that is an immune checkpoint molecule. In some embodiments, the second antibody comprises a specificity for an immune checkpoint molecule selected from PD1, LAG3, CTLA-4, A2AR, TIM-3, BTLA, CD276, CD328, VTCN1, IDO, KIR, NOX2, VISTA, OX40, CD27, CD28, CD40, CD122, CD137, GITR, ICOS. In at least one embodiment, the pharmaceutical composition comprising an anti-PD-L1 antibody and an additional active agent, wherein the additional active agent is an antibody comprising a specificity for the immune checkpoint molecule PD1. Exemplary antibodies comprising a specificity for PD1 that are useful in the pharmaceutical composition embodiments disclosed herein include, but are not limited to, pembrolizumab, nivolumab, cemiplimab, pidilizumab, dostarlimab, and HX008.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, the formulation can be a sustained-release preparation of the antibody and/or other active ingredients. Suitable examples of sustained-release preparations include semipermeable matrices of solid hydrophobic polymers containing the antibody, which matrices are in the form of shaped articles, e.g. films, or microcapsules.

Typically, the formulations of the present disclosure to be administered to a subject are sterile. Sterile formulations may be readily prepared using well-known techniques, e.g., by filtration through sterile filtration membranes.

### Modes for Administering Anti-PD-L1 Antibody-IL-10 Fusion

Administration to a subject in need of a composition or formulation comprising an anti-PD-L1 antibody-IL-10 fusion in accordance with the methods of treatment provides a therapeutic effect that protects the subject from and/or treats cancer, cancer recurrence or virus infection via promoting T cell memory response.

In some embodiments of the methods of treatment of the present disclosure, the anti-PD-L1 antibody-IL-10 fusion composition or formulation comprising an anti-PD-L1 antibody-IL-10 fusion is administered to a subject by any mode of administration that delivers the agent systemically, or to a desired target tissue. Systemic administration generally refers to any mode of administration of the antibody into a subject at a site other than directly into the desired target site, tissue, or organ, such that the antibody or formulation thereof enters the subject's circulatory system and, thus, is subject to metabolism and other like processes.

Accordingly, modes of administration useful in the methods of treatment of cancer, cancer recurrence or virus infection via promoting T cell memory response of the present disclosure can include, but are not limited to, injection, infusion, instillation, and inhalation. Administration by injection can include intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion.

In an embodiment according to the methods of the present disclosure, the anti-PD-L1 antibody-IL-10 fusion may be administered to the subject in need thereof by at least one route selected from the group consisting of parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal. In one embodiment, the anti-PD-L1 antibody-IL-10 may be administered to the subject in need intravenously.

In some embodiments, a formulation of the anti-PD-L1 antibody-IL-10 is formulated such that the antibody is protected from inactivation in the gut. Accordingly, the method of treatments can comprise oral administration of the formulation.

In some embodiments, the present disclosure provides uses of compositions or formulations comprising an anti-PD-L1 antibody-IL-10 as a medicament for the treatment of cancer, cancer recurrence or virus infection via promoting T cell memory response. Additionally, in some embodiments, the present disclosure also provides for the use of a composition or a formulation comprising an anti-PD-L1 antibody-IL-10 in the manufacture or preparation of a medicament for the treatment of cancer, cancer recurrence or virus infection via promoting T cell memory response. In a further embodiment, the medicament is for use in a method for treating cancer, cancer recurrence or virus infection via promoting T cell memory response comprising administering to a subject in need thereof an effective amount of the medicament. In certain embodiments, the medicament further comprises an effective amount of at least one additional therapeutic agent, or treatment.

In a further embodiment, the medicament is for use in treating cancer, cancer recurrence or virus infection via promoting T cell memory response in a subject comprising administering to the subject an amount effective of the medicament to treat the cancer, cancer recurrence or virus infection via promoting T cell memory response.

For the treatment of a cancer, cancer recurrence or virus infection via promoting T cell memory response, the appropriate dosage of the anti-PD-L1 antibody-IL-10 contained in the compositions and formulations of the present disclosure (when used alone or in combination with one or more other additional therapeutic agents) will depend on factors including the severity and course of the disease, whether the antibody is administered for preventive or therapeutic purposes, the previous therapy administered to the patient, the patient's clinical history and response to the antibody, and the discretion of the attending physician.

Generally, a treatment regimen useful in the methods of the present disclosure can be decided by the medical personnel of the subject in need. The anti-PD-L1 antibody-IL-10 of the present disclosure when included in the compositions and formulations described herein, can be suitably administered to the patient at one time, or over a series of treatments. Various dosing schedules including but not limited to single or multiple administrations over various time-points, bolus administration, and pulse infusion are contemplated herein.

In some embodiments of the methods, the composition comprising an anti-PD-L1 antibody-IL-10 may be administered to the subject in need more than once a day, at least once a day, at least once a week, or at least once a month.

Depending on the type and severity of the cancer, cancer recurrence or virus infection, about 1 μg/kg to 15 mg/kg of anti-PD-L1 antibody-IL-10 in a formulation of the present disclosure is an initial candidate dosage for administration to a human subject, whether, for example, by one or more separate administrations, or by continuous infusion. Generally, the administered dosage of the antibody would be in the range from about 0.05 mg/kg to about 10 mg/kg. In some embodiments, one or more doses of about 0.5 mg/kg, 2.0 mg/kg, 4.0 mg/kg or 10 mg/kg (or any combination thereof) may be administered to a patient.

Dosage administration can be maintained over several days or longer, depending on the condition of the subject, for example, administration can continue until the cancer, cancer recurrence or virus infection is sufficiently treated, as determined by methods known in the art. In some embodiments, an initial higher loading dose may be administered, followed by one or more lower doses. However, other dosage regimens may be useful. The progress of the therapeutic effect of dosage administration can be monitored by conventional techniques and assays.

Accordingly, in some embodiments of the methods of the present disclosure, the administration of the anti-PD-L1 antibody-IL-10 comprises a daily dosage from about 1 mg/kg to about 100 mg/kg. In some embodiments, the dosage of anti-PD-L1 antibody-IL-10 comprises a daily dosage of at least about 1 mg/kg, at least about 5 mg/kg, at least about 10 mg/kg, at least about 20 mg/kg, or at least about 30 mg/kg.

Combination Treatment Methods with Other Therapeutic Agents

In an embodiment according to the present invention, the method may further comprise the step of administering at least one additional therapeutic agent. For example, the additional therapeutic agent may be administered to the subject in need thereof in combination with the anti-PD-L1 antibody-IL-10 composition—e.g., administered at the same time as the anti-PD-L1 antibody-IL-10 composition; before administration of the anti-PD-L1 antibody-IL-10 composition; or after administration of the anti-PD-L1 antibody-IL-10 composition. In some embodiments, the additional therapeutic agent may comprise an additional treatment for cancer, cancer recurrence or virus infection or a treatment for a disease or condition associated with cancer, cancer recurrence or virus infection. It is contemplated that in the combination treatment method, by administering an anti-PD-L1 antibody-IL-10 composition in combination with a therapeutic agent the efficacy of the therapeutic agent may be improved. Without being bound by theory, it is believed that after administering a therapeutically effective amount of the anti-PD-L1 antibody-IL-10 composition and an optional additional therapeutic agent, an immune response against cancer, cancer recurrence or virus infection may be induced or boosted in the subject in need; size or severity of the cancer may be reduced; seroconversion with respect to virus may be induced; or a viral load of virus in the subject in need may be reduced, even to a level when it is undetectable. Additionally, the boosted immune response may comprise production of antibodies or cytokines that further modulate the activity of the immune system.

In one embodiment, the method comprising administering at least one additional therapeutic agent is carried out wherein the additional therapeutic agent is a virus vaccine. As described elsewhere herein, the virus vaccines useful in the combination treatment method can include virus vaccines for prophylaxis as well as therapeutic vaccines for treatment of subjects already infected with the virus. In some embodiments, the therapeutic vaccine is selected from a DNA vaccine, a viral vector vaccine, a protein vaccine, and a multi-peptide vaccine.

As described elsewhere herein, inhibitory immune checkpoint molecules are targets for treatment of some viral infections as well as cancer. Accordingly, in one embodiment, the method comprising administering at least one additional therapeutic agent is carried out wherein the additional therapeutic agent is an antibody that targets an inhibitory immune checkpoint molecule. In some embodiments, the inhibitory immune checkpoint molecule is selected from PD1, PD-L1, and CTLA-4. In some embodiments, the antibody that targets an inhibitory immune checkpoint molecule is selected from an anti-PD1, anti-PD-L1, and an anti-CTLA-4.

In other embodiments according to the present invention, the additional therapeutic agent may be selected from the group consisting of: a therapeutic agent; an imaging agent; a cytotoxic agent; an angiogenesis inhibitor; a kinase inhibitor; a co-stimulation molecule blocker; an adhesion molecule blockers; an anti-cytokine antibody or functional fragment thereof; methotrexate; cyclosporin; rapamycin; FK506; a detectable label or reporter; a TNF antagonist; an anti-rheumatic; a muscle relaxant; a narcotic; a non-steroid anti-inflammatory drug (NSAID); an analgesic; an anesthetic; a sedative; a local anesthetic; a neuromuscular blocker; an antimicrobial; an antipsoriatic; a corticosteroid; an anabolic steroid; an erythropoietin; an immunization; an immunoglobulin; an immunosuppressive; a growth hormone; a hormone replacement drug; a radiopharmaceutical; an antidepressant; an antipsychotic; a stimulant; an asthma medication; a beta agonist; an inhaled steroid; an epinephrine or analog thereof; a cytokine; and a cytokine antagonist; an immunomodulatory agent.

In some embodiments, the additional therapeutic agent is selected from a viral entry inhibitor, a viral RNA inhibitor, a gene editing agent, a viral antigen secretion inhibitors, a polymerase inhibitor, an interferon, a viral maturation inhibitor, a nucleoside reverse transcriptase inhibitor, a capsid assembly inhibitor/modulator, a cccDNA inhibitor, an FXR agonist, a microRNA, a TLR agonist, and an immunomodulators.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Generation of Bifunctional Anti-PDL1/IL-10 Fusion Proteins

### A. Recombinant Anti-PDL1/IL-10 Fusion Protein Production

Recombinant IL10-Fc fusion protein was designed by genetically fusing human IL-10 to the N-terminus of the human IgG1-Fc separated by a 14 amino acid linker. Recombinant anti-PDL1/IL-10 fusion proteins were designed by genetically fusing human IL-10 (WT, R107A or R104Q/R107A) to the C-terminus of the anti-PDL1 (Atezolizumab, Avelumab, Durvalumab, PHS102, HSYPP31F, HSYPP411C, YP7G, YP11F, YT6D, YT7A, YT7H, or YT10H) IgG1 antibody fragments separated by a 14 amino acid linker. The desired gene segments, preceded by an IL-2 secretion sequence required for secretion of recombinant proteins, were obtained using Thermo gene synthesis service and cloned in a mammalian expression vector. The fusion proteins were expressed in transfected ExpiCHO cells.

### B. Recombinant Antibody Purification

Antibodies were transiently expressed in ExpiCHO-S cells (Thermo Scientific). During exponential growing phage, 6×10⁶ ExpiCHO-S cells were transiently transfected with 20 μg of the vectors encoding IL10-Fc or anti-PDL1/IL-10 fusion proteins by ExpiFectamine CHO Transfection Kit (Thermo Scientific). 18-22 hours after transfection, ExpiFectamine CHO Enhancer and ExpiCHO Feed were added to the flask. The cells were cultured for 8 days. The supernatant of each culture was centrifuged and subsequently filtered through a 0.45 μm filter.

Antibodies were purified from transfected cell supernatants with Protein A Sepharose Fast Flow beads (GE Healthcare). Antibody loaded columns were washed with PBS, and then eluted with 0.1 M Glycine (pH 2.5) directly into 1/10 volume of 1M Tris buffer (pH 9.0). Antibody containing fractions were pooled and dialyzed against PBS. The quality of purified antibodies was examined by SDS-PAGE in the presence and absence of a reducing agent.

Details about the generation, affinity maturation and characterization of anti-PD-L1 antibody fusion can be found in the examples appended to PCT publication no. WO2021231741A2, which is incorporated herein by reference.

### Example 2: Anti-PDL1/IL-10 Fusion Proteins Enhance Antigen Presenting Cell (APC)-Mediated T-Cell Activation in Mix Lymphocyte Reaction (MLR)

This example illustrates studies of the ability of anti-PDL1/IL-10 to enhance T cell activation in MLR. The increased production of the IFNγ is known to be associated with T cell effector function.

Materials and methods: Human peripheral blood was obtained from healthy donors. Peripheral blood mononuclear cells (PBMC) were immediately isolated by density gradient centrifugation using Ficoll-Paque Plus (GE Healthcare). To serve as allogeneic APCs, CD14+ monocytes were first isolated by using anti-human CD14 conjugated magnetic beads (Miltenyi Biotec) from Donor A. For immature dendritic cell (DC) generation, monocytes were cultured with GM-CSF (20 ng/ml) and IL-4 (20 ng/ml) in RPMI1640 supplemented with 10% FBS for 6 days. For mature DC generation, immature DCs were treated with LPS (500 ng/mL) for 24 hr. Mature DCs were treated with 40 μg/mL mitomycin C at 37° C. for 30 min before co-culture with T cells.

CD4+ T cells were isolated by using anti-human CD4 conjugated magnetic beads (Miltenyi Biotec) from Donor B. Responder CD4 T cells were resuspended at 4×10⁶ cells/mL in culture medium, and 50 μL of T cells were added to all wells with the exception of the DC-only wells. Stimulator DCs were resuspended at 4×10⁶ cells/mL in culture medium, and 50 μL of DCs were added to all wells with the exception of the CD4 T-only wells. An additional 100 μL of culture medium containing 0.1-2 μg IL10-Fc or anti-PDL1/IL-10 (Avelumab/IL10, HSYPP411C/IL10, YP7G/IL10, YP11F/IL10, YT7A/IL10, YT7H/IL10 or YT10H/IL10) fusion proteins were added to the CD4 T-DC culture in a 96-well U-bottom plate. The co-cultures were incubated at 37° C. IFN-γ levels released in the supernatants are measured after 5 days by ELISA (Biolegend) according to the manufacturer's instructions.

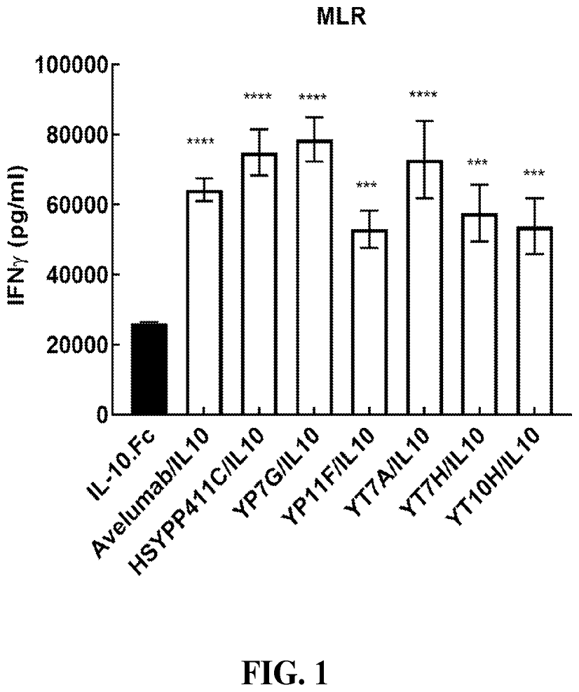

Results: As shown in FIG. 1, the exemplary anti-PDL1/IL-10 fusion proteins are capable of enhancing T cell activation relative to IL10-Fc controls in MLR assay.

### Example 3: Accumulation of Anti-PDL1/IL-10 Fusion Protein in the Tumor

This example illustrates a study of the ability of anti-PDL1/IL-10 to accumulate within tumor tissue in HNSC model.

Materials and Methods: Purified anti-PDL1/IL-10 fusion protein (Avelumab/IL10) was labeled using a near-infrared (NIR) fluorochrome labeling Kit (VivoTag 680XL, PerkinElmer) according to the manufacturer's instructions. HNSC/Q1-2^{Lucferiease} is a luciferase-expressing subline from parental MTC-Q1 (mouse oral cancer cell line), which was kindly provided by Dr. Kuo-Wei Chang (Department of Dentistry, National Yang Ming Chiao Tung University). HNSC/Q1-2^{Lucferiease} tumor cells were subcutaneously inoculated into syngeneic C56BL/6 mice for tumor growth. After a 2-week growth period, HNSC/Q1-2^{Lucferiease} tumor-bearing mice were intravenously injected with 150 μg VivoTag680-labeled anti-PDL1/IL-10 (Avelumab/IL10) for 24 h followed by in vivo bioluminescence and fluorescence detection using a Xenogen IVIS 100 imaging system. For the biodistribution of anti-PDL1/IL10, mouse tissues with VivoTag680 signals, including tumor and spleen were isolated to confirm the existence of anti-PDL1/IL-10 by fluorescence examination with a Vectra Polaris Imaging system (Akoya Biosciences).

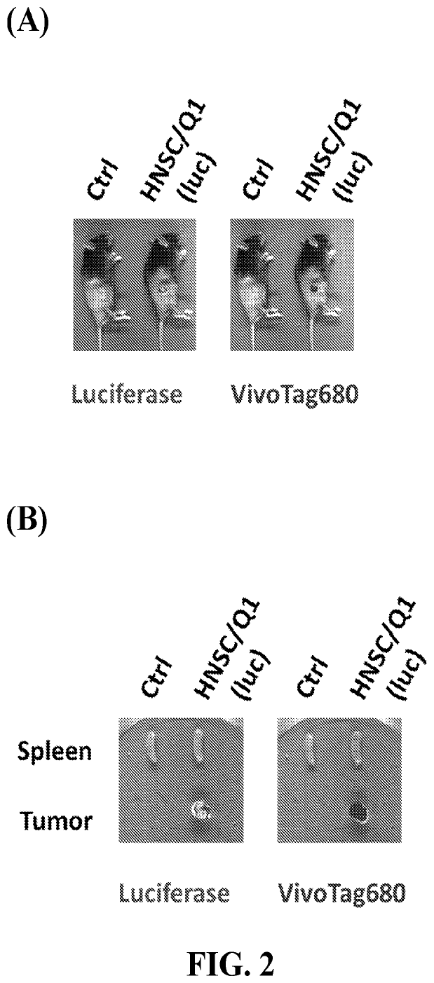

Results: As shown in FIG. 2, anti-PDL1/IL-10 accumulates specifically in the tumor but not spleen or other tissue after i.v. administration.

### Example 4: Anti-PDL1/IL-10 Enhances T Cell Response In Vivo

This example illustrates studies of the ability of anti-PDL1/IL-10 treatment to increase the serum level of cytokines related to T cell response.

Materials and methods: BALB/c mice (6-8 weeks old) were implanted subcutaneously with 5×10⁵ CT26 cells (ATCC CRL-2638). After 8 days, mice were randomized into treatment groups when tumor volume reached 50-100 mm³. Mice were then injected intraperitoneally (i.p.) twice weekly for 3 weeks with PBS control, 3 mg/kg IL10-Fc (92 kDa), 6 mg/kg anti-PDL1/TGFbR (Avelumab/TGFβR2, M7824, 177 kDa), or 6 mg/kg anti-PDL1/IL-10 (Avelumab/IL10, 185.5 kDa). The blood samples were collected on Day29. Serum level of mouse CXCL9 and IL-18 were measured by ELISA kits (Biolegend).

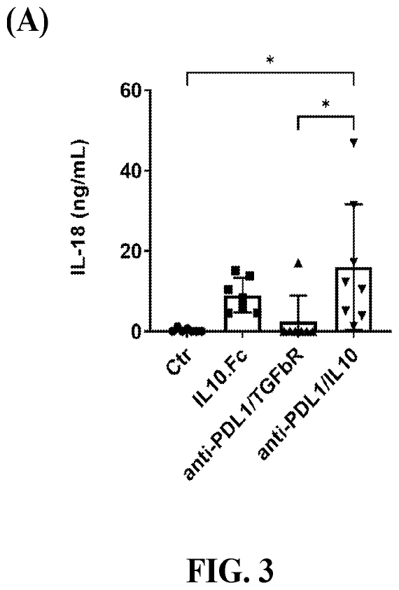

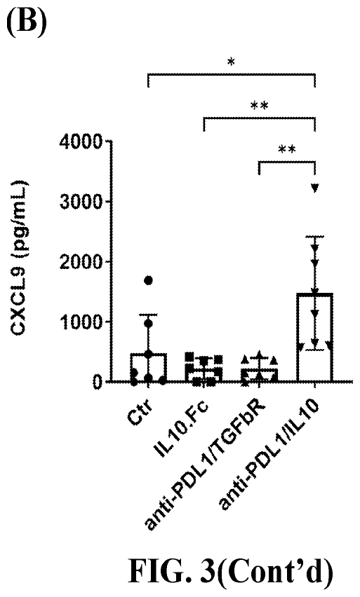

Results: As shown in the FIG. 3, serum level of IL-18 were greatly increased in the anti-PDL1/IL-10 group comparing to the control group and the anti-PDL1/TGFbR group. In addition, serum level of CXCL9 was also greatly increased comparing to other three groups.

### Example 5: The Ability of Anti-PDL1/IL-10 to Enhance Tumor Control was Dependent on Increased Activity of Tumor-Resident Cells

This example illustrates a study of the dependency of tumor-resident cells to anti-PDL1/IL-10 mediated tumor control.

Materials and methods: BALB/c mice (6-8 weeks old) were implanted subcutaneously with 5×10⁵ EMT6 cells (ATCC CRL-2755). After 10 days, mice were randomized into treatment groups when tumor volume reached 50-100 mm³. EMT6 tumor-bearing mice (n=6) were treated with vehicle (1% ethanol, PO) or FTY720 (2 mg/kg, PO) daily for 2 weeks. PBS, anti-PDL1 (Avelumab) (10 mg/kg) or anti-PDL1/IL-10 (Avelumab/IL10) (12 mg/kg) were administered via i.p injection twice weekly from day 11 for 4 weeks. Tumor volume was measured twice per week by caliper measurements until end of the study.

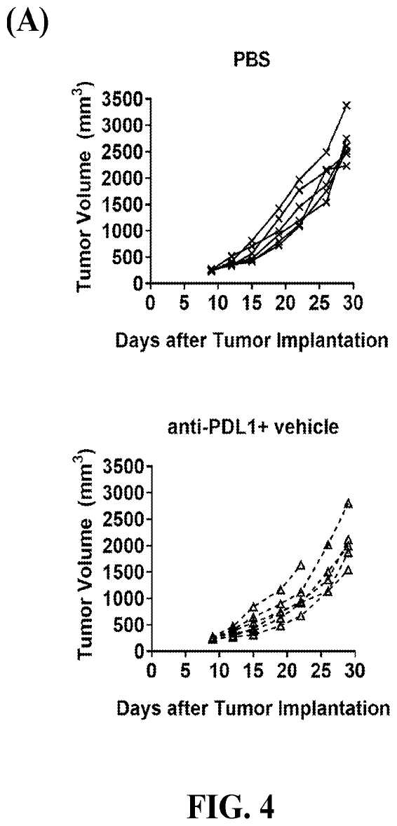

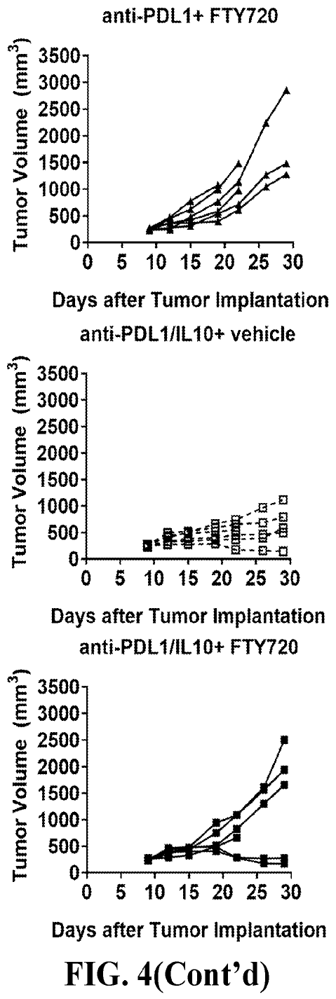

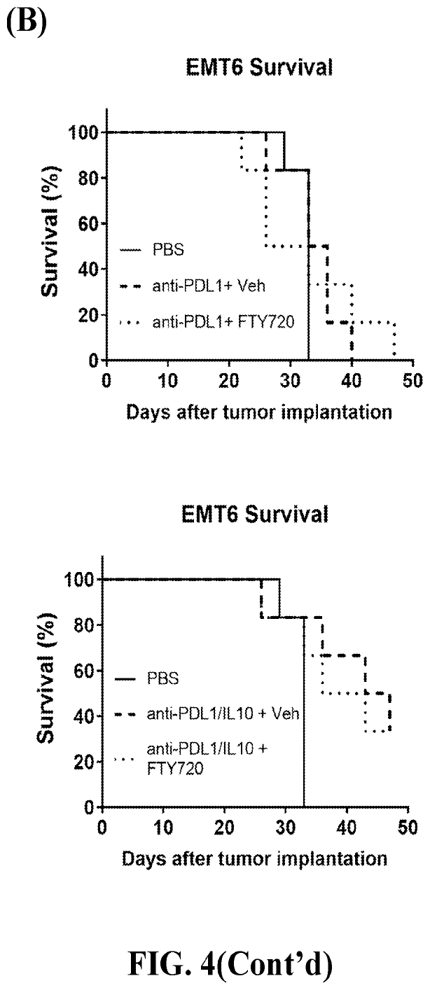

Results: As shown in FIG. 4, in the anti-PDL1/IL-10 group, tumor volume was smaller and survival rate was higher comparing to the anti-PDL1 group, indicating that anti-PDL1/IL-10 is important for the tumor control. Co-treatment with FTY720, which inhibits lymphocyte egress from secondary lymphoid organs, did not reduce anti-tumor activity of the anti-PDL1/IL-10 in some albeit not all mice.

### Example 6: Anti-PDL1/IL-10 Treatment Generates Durable T Cell Memory Protection

This example illustrates a study of the ability of anti-PDL1/IL-10 to generate a durable anti-tumor immune response.

Materials and methods: BALB/c mice (6-8 weeks old) were implanted subcutaneously in the right rear flank region with 5×10⁵ EMT6 cells (ATCC CRL-2755). After 7 days, mice were randomized into treatment groups when tumor volume reached 50-100 mm³. Mice (n=7) were then injected intraperitoneally twice weekly for 3 weeks with PBS, anti-PDL1 (5 mg/kg), anti-PDL1/IL-10 (6 mg/kg) or anti-PDL1/TGFbR (6 mg/kg). For rechallenge study, 5 weeks after tumor ‘cure’ (day 67), cured and naïve Balb/c mice (n=5) were implanted subcutaneously in the left rear flank region with EMT6 tumor cells. Tumor volume was measured twice per week by caliper measurements until end of the study.

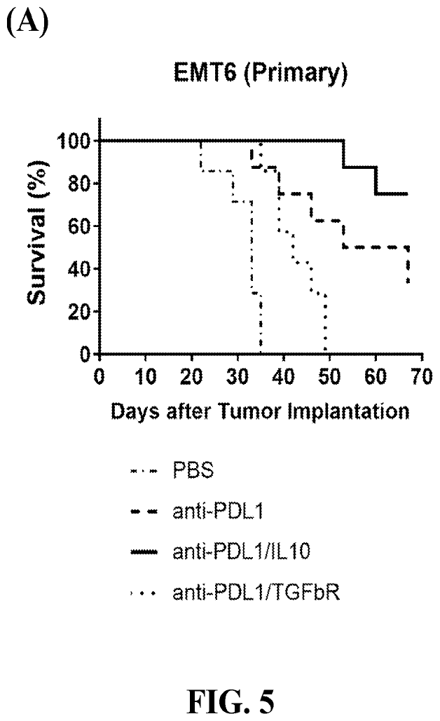

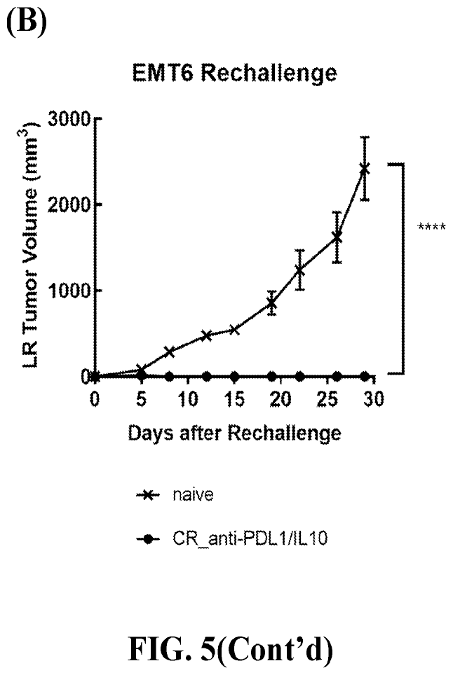

Results: As shown in FIG. 5 and Table 3, the anti-PDL1/IL-10 treatment showed higher survival rate and tumor growth inhibition comparing to the anti-PDL1 treatment and the anti-PDL1/TGFbR treatment. Similarly, for rechallenge study, anti-PDL1/IL10-treated mice which were tumor-free for 5 weeks were challenged with EMT6 cells. No mice grew new tumors over the next month compared to 100% tumor formation in naïve mice, indicating anti-PDL1/IL10 could elicit durable tumor-specific T cell response and memory protection.

TABLE 3
Summary of tumor growth inhibition, numbers of cured
mice and median overall survival in EMT6 tumor study.

| | | Anti- | Anti- | Anti- |
|---|---|---|---|---|
| (Day 32) | PBS | PDL1 | PDL1/IL10 | PDL1/TGFbR |
| Tumor Growth Inhibition | — | 67% | 97% | 53% |
| # Cured | 0/7 | 1/7 | 5/7 | 0/7 |
| % Cured | 0% | 14% | 71% | 0% |
| Median Survival (days) | 33 | 60 | ND | 42 |

### Example 7: Anti-PDL1/IL-10 Muteins Control Tumor Growth and Enhance T Cell Response

This example illustrates the studies of the ability of anti-PDL1/IL-10 mutants to control tumor growth and enhance T cell response.

Materials and methods: BALB/c mice (6-8 weeks old) were implanted subcutaneously with 5×10⁵ CT26 cells (ATCC CRL-2638). After 7 days, mice were randomized into treatment groups when tumor volume reached 50-100 mm³. Mice were then injected intraperitoneally twice weekly for 3 weeks with 1 mg/kg anti-PDL1/IL-10 or anti-PDL1/IL-10 mutants (R107A, R104Q/R107A). Serum level of mouse CXCL9 were measured by ELISA kits (Biolegend)

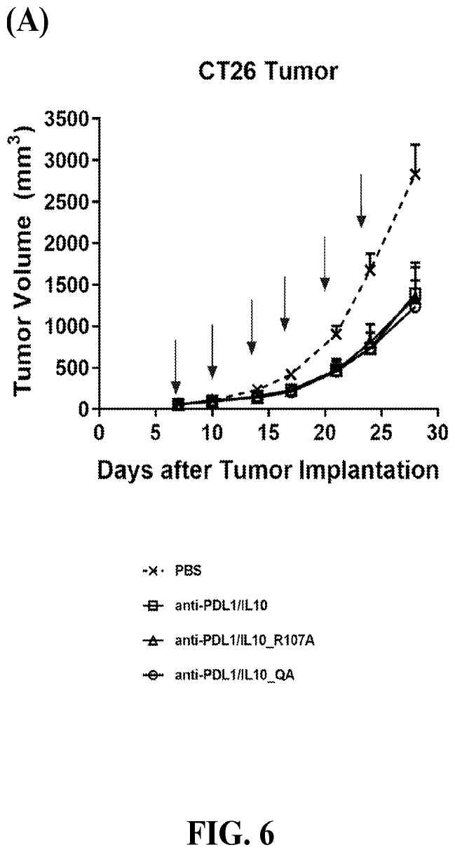

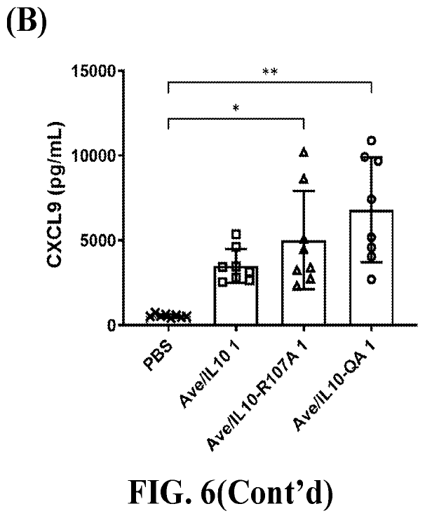

Results: As shown in FIG. 6, anti-PDL1/IL-10 muteins, anti-PDL1/IL-10_R107A and anti-PDL1/IL-10_QA showed similar tumor growth curve as anti-PDL1/IL-10 WT. In addition, serum level of CXCL9 was also greatly increased comparing to the control group, indicating that the T cell response was enhanced in the anti-PDL1/IL-10 group, either with IL-10 WT or IL-10 muteins, comparing to the control group.

### Example 8: Tumor-Infiltrating Lymphocytes (TILs) Isolated from Anti-PDL1/IL10-Treated Tumor-Bearing Mice Exhibit Better Recall Responses

This example illustrates PEXs isolated from anti-PDL1/IL10-treated tumor-bearing mice exhibit better recall responses.

Materials and methods: C57BL/6 mice were inoculated subcutaneously with 8×10⁵ YUMM1.7-GP33 tumor cells on Day 0, followed by i.v. adoptive transfer of 2×10⁶ early activated Tcf7^{GFP} P14 T cells on Day 9 post engraftment. The P14 TCR-expressed T cells are specific for the gp33 epitope of lymphocytic choriomeningitis virus (LCMV). Tumor-bearing mice were further i.p. administrated with PBS or 125 μg anti-PDL1/IL-10 (Avelumab/IL10) on Day 10 and Day 14. After harvesting the tumors on Day 15, progenitor exhausted CD8⁺ TILs were further sorted out based on the markers, TCF7-GFP⁺PD-1⁺TIM3⁻, followed by transferring into naïve recipient mice. Next day, TIL-transferred mice were further rechallenged with LCMV-Arm (2×10⁵ CFU). Lastly, the spleens were collected, and the transferred cells were analyzed on Day 8 post infection.

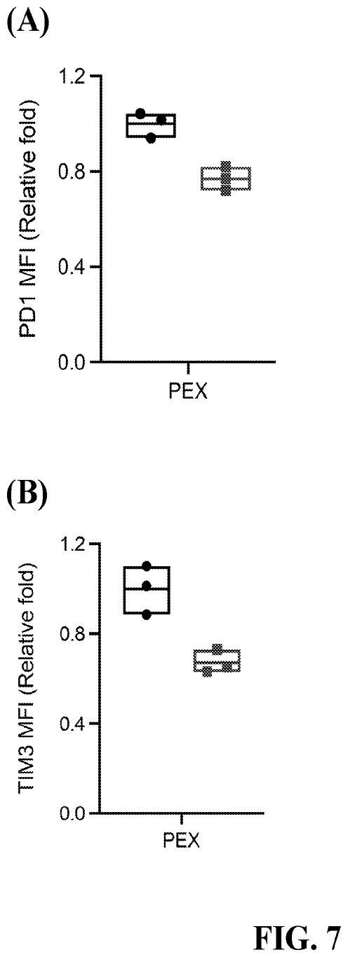

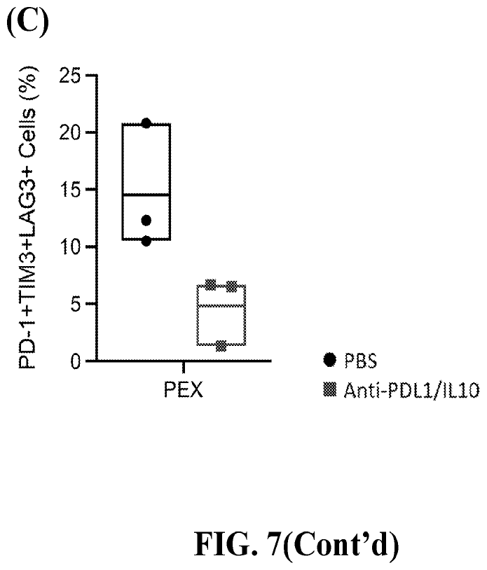

Results: FIG. 7 depicts that, upon rechallenge, TILs treated with anti-PDL1/IL-10 display better immune qualities with decreased expression level of co-inhibitory receptors of (A) PD1 and (B) TIM3, and (C) decreased PD-1⁺TIM3⁺LAG3⁺ cell population in comparison with PBS-treated group.

### Example 9: Anti-PDL1/IL-10 Treatment Boosts Progenitor Exhausted T Cells (PEX)-Dependent Durable Tumor Protection

This example illustrates anti-PDL1/IL-10-boosted durable tumor protection is PEX-dependent.

Materials and methods: C57BL/6 mice were inoculated subcutaneously with 8×10⁵ YUMM1.7-GP33 tumor cells on Day 0, followed by i.v. adoptive transfer of early activated 4×10⁶ Tcf7DTR-GFP P14 T cells on Day 6 post engraftment. Tumor-bearing mice were further i.p. administrated with 125 μg anti-PDL1/IL-10 (Avelumab/IL10) every three days starting on Day 8 until tumors became undetectable. Three weeks later, tumor-free mice were either i.p. injected three times with PBS or 1.5 μg diphtheria toxin (DT) to eliminate TCF1+ diphtheria toxin receptor (DTR)-expressing cells within 1 week. Lastly, all the tumor-free mice were s.c. inoculated with 2×10⁵ B16-GP33 melanoma cells and the tumor growth was monitored every other day. Age-matched naïve mice were also inoculated with B16-GP33 cells as a positive control for tumor progression.

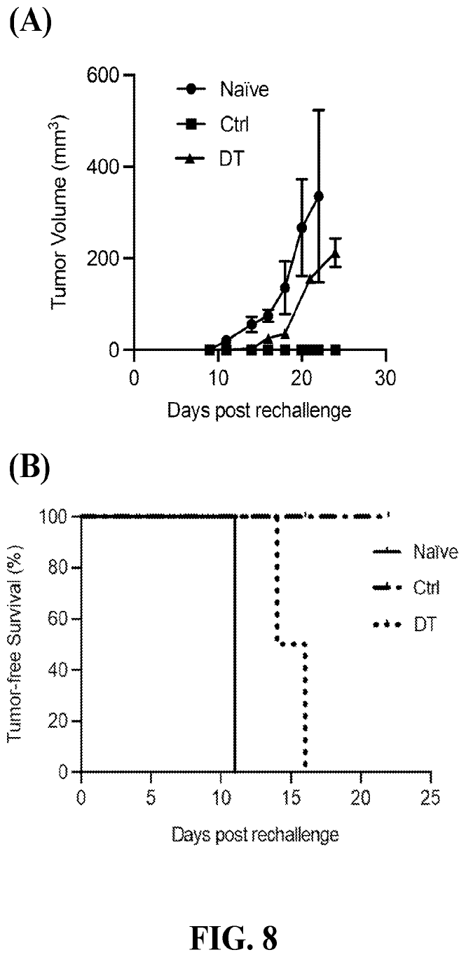

Results: As shown in FIG. 8, age-matched naïve mice were inoculated with B16-GP33 melanoma cells as a positive control (Naïve) for tumor progression. (A) Tumor growth and (B) tumor-free survival were monitored for 4 weeks. The control group (Ctrl) of tumor-free mice without elimination of progenitor exhausted T cells (PEX) remains tumor-free status, whereas B16-GP33 tumor lesions can be observed on Day 14-16 post rechallenge in DT-treated group, indicating that anti-PDL1/IL10-induced durable protection is PEX-dependent.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims. Additional embodiments of the invention are set forth in the following claims.

What is claimed is:

1. A method for promoting T cell memory response in a subject in need thereof, treating cancer in a subject in need thereof, preventing cancer recurrence in a subject in need thereof, or treating chronic virus infection diseases in a subject in need thereof, comprising the step of:
administering a composition comprising a therapeutically effective amount of a PD-L1 antibody-IL-10 fusion protein and a pharmaceutically acceptable carrier.

2-19. (canceled)

20. The method of claim 1, wherein the PD-L1 antibody comprises a heavy chain (HC) and the HC is fused via a linker to the IL10; and optionally, wherein the linker comprises an amino acid sequence selected from SEQ ID NO: 74, 75, 76, 77, 78, and 79.

21. The method of claim 1, wherein:
(a) the IL-10 comprises an amino acid sequence of SEQ ID NO: 73, 172, 173, 174, 175, 176, 177, 178, 179 or 180;
(b) the IL-10 is a naturally-occurring or engineered variant of IL-10 that retains its cytokine activity;
(c) the IL-10 is a synthetically modified version of IL-10 that retains its cytokine activity; and/or
(d) the IL-10 comprises one, two, or four IL-10 polypeptides.

22. The method of claim 1, wherein the IL-10 comprises a substitution on amino acids in position 104, position 107, and a combination thereof, relative to amino acids of wild-type IL-10.

23. The method of claim 22, wherein the wild-type IL-10 comprises the amino acid sequence having at least 80%, preferably at least 90%, and more preferably at least 95%, identity with SEQ ID NO: 73, 172, 173, 174, 175, 176, 177, 178, 179 or 180.

24. The method of claim 23, wherein the substitution comprises:
(a) R104Q;
(b) any one of R107A, R107E, R107Q and R107D; or
(c) a combination thereof.

25. The method of claim 24, wherein the substitution comprises R104Q/R107A, R104Q/R107E, R104Q/R107Q or R104Q/R107D.

26. The method of claim 1, wherein the PD-L1 antibody comprises (i) a first light chain complementary determining region (CDR-L1), a second light chain complementary determining region (CDR-L2), and a third light chain complementary determining region (CDR-L3), and (ii) a first heavy chain complementary determining region (CDR-H1), a second heavy chain complementary determining region (CDR-H2), and a third heavy chain complementary determining region (CDR-H3), wherein:
(a) CDR-H1 comprises an amino acid sequence selected from SEQ ID NO: 92, and 123;
(b) CDR-H2 comprises an amino acid sequence selected from SEQ ID NO: 109, 114, and 124;
(c) CDR-H3 comprises an amino acid sequence selected from SEQ ID NO: 94, and 110;
(d) CDR-L1 comprises an amino acid sequence selected from SEQ ID NO: 53;
(e) CDR-L2 comprises an amino acid sequence selected from SEQ ID NO: 54;
(f) CDR-L3 comprises an amino acid sequence selected from SEQ ID NO: 55, 112, and 116.

27. The method of claim 26, wherein:
(a) CDR-H1 comprises the amino acid sequence of SEQ ID NO: 92, CDR-H2 comprises the amino acid sequence of SEQ ID NO: 109, CDR-H3 comprises the amino acid sequence of SEQ ID NO: 110, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 112;
(b) CDR-H1 comprises the amino acid sequence of SEQ ID NO: 92, CDR-H2 comprises the amino acid sequence of SEQ ID NO: 114, CDR-H3 comprises the amino acid sequence of SEQ ID NO: 94, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 116; or
(c) CDR-H1 comprises the amino acid sequence of SEQ ID NO: 123, CDR-H2 comprises the amino acid sequence of SEQ ID NO: 124, CDR-H3 comprises the amino acid sequence of SEQ ID NO: 94, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 55.

28. The method of claim 26, wherein the antibody comprises a heavy chain variable domain (VH) amino acid sequence having at least 90% identity to a sequence selected from SEQ ID NO: 111, 115, and 125; and/or a light chain variable domain (VL) amino acid sequence having at least 90% identity to a sequence selected from SEQ ID NO: 56, 113, and 117; optionally, wherein:
(a) the antibody comprises a VH amino acid sequence having at least 90% identity to SEQ ID NO: 111; and a VL amino acid sequence having at least 90% identity to SEQ ID NO: 113;
(b) the antibody comprises a VH amino acid sequence having at least 90% identity to SEQ ID NO: 115; and a VL amino acid sequence having at least 90% identity to SEQ ID NO: 117; or
(c) the antibody comprises a VH amino acid sequence having at least 90% identity to SEQ ID NO: 125; and a VL amino acid sequence having at least 90% identity to SEQ ID NO: 56.

29. The method of claim 26, wherein the antibody comprises a heavy chain (HC) amino acid sequence having at least 90% identity to a sequence selected from SEQ ID NO: 157, 158, and 160, and/or a light chain (LC) amino acid sequence having at least 90% identity to a sequence selected from SEQ ID NO: 135, 137, and 141; optionally, wherein the antibody comprises:
(a) the HC amino acid sequence of SEQ ID NO: 157, and the LC amino acid sequence of SEQ ID NO: 135;
(b) the HC amino acid sequence of SEQ ID NO: 158, and the LC amino acid sequence of SEQ ID NO: 137;
(c) the HC amino acid sequence of SEQ ID NO: 160, and the LC amino acid sequence of SEQ ID NO: 141.

30. The method of claim 26, wherein the antibody comprises a heavy chain (HC) fused via a linker to an IL-10 polypeptide, wherein the HC-IL-10 fusion amino acid sequence has at least 90% identity to a sequence selected from SEQ ID NO: 134, 136, and 140; and a light chain (LC) having an amino acid sequence of at least 90% identity to a sequence selected from SEQ ID NO: 135, 137, and 141; optionally, wherein the antibody comprises:
(a) the HC-IL-10 fusion amino acid sequence of SEQ ID NO: 134, and the LC amino acid sequence of SEQ ID NO: 135;
(b) the HC-IL-10 fusion amino acid sequence of SEQ ID NO: 136, and the LC amino acid sequence of SEQ ID NO: 137; or
(c) the HC-IL-10 fusion amino acid sequence of SEQ ID NO: 140, and the LC amino acid sequence of SEQ ID NO: 141.

31. The method of claim 1, wherein the PD-L1 antibody comprises a heavy chain (HC) fused via a linker to the IL10, wherein the antibody comprises (i) a first light chain complementary determining region (CDR-L1), a second light chain complementary determining region (CDR-L2), and a third light chain complementary determining region (CDR-L3), and (ii) a first heavy chain complementary determining region (CDR-H1), a second heavy chain complementary determining region (CDR-H2), and a third heavy chain complementary determining region (CDR-H3), wherein:
(a) CDR-H1 comprises the amino acid sequence of SEQ ID NO: 1, CDR-H2 comprises the amino acid sequence of SEQ ID NO: 2, CDR-H3 comprises the amino acid sequence of SEQ ID NO: 3, CDR-L1 comprises the amino acid sequence of SEQ ID NO: 5, CDR-L2 comprises the amino acid sequence of SEQ ID NO: 6, and CDR-L3 comprises the amino acid sequence of SEQ ID NO: 7;
(b) CDR-H1 comprises the amino acid sequence of SEQ ID NO: 9, CDR-H2 comprises the amino acid sequence of SEQ ID NO: 10, CDR-H3 comprises the amino acid sequence of SEQ ID NO: 11, CDR-L1 comprises the amino acid sequence of SEQ ID NO: 13, CDR-L2 comprises the amino acid sequence of SEQ ID NO: 14, and CDR-L3 comprises the amino acid sequence of SEQ ID NO: 15;
(c) CDR-H1 comprises the amino acid sequence of SEQ ID NO: 17, CDR-H2 comprises the amino acid sequence of SEQ ID NO: 18, CDR-H3 comprises the amino acid sequence of SEQ ID NO: 19, CDR-L1 comprises the amino acid sequence of SEQ ID NO: 21, CDR-L2 comprises the amino acid sequence of SEQ ID NO: 22, and CDR-L3 comprises the amino acid sequence of SEQ ID NO: 23;
(d) CDR-H1 comprises the amino acid sequence of SEQ ID NO: 25, CDR-H2 comprises the amino acid sequence of SEQ ID NO: 26, CDR-H3 comprises the amino acid sequence of SEQ ID NO: 27, CDR-L1 comprises the amino acid sequence of SEQ ID NO: 29, CDR-L2 comprises the amino acid sequence of SEQ ID NO: 30, and CDR-L3 comprises the amino acid sequence of SEQ ID NO: 31;
(e) CDR-H1 comprises the amino acid sequence of SEQ ID NO: 33, CDR-H2 comprises the amino acid sequence of SEQ ID NO: 34, CDR-H3 comprises the amino acid sequence of SEQ ID NO: 35, CDR-L1 comprises the amino acid sequence of SEQ ID NO: 37, CDR-L2 comprises the amino acid sequence of SEQ ID NO: 38, and CDR-L3 comprises the amino acid sequence of SEQ ID NO: 39;
(f) CDR-H1 comprises the amino acid sequence of SEQ ID NO: 41, CDR-H2 comprises the amino acid sequence of SEQ ID NO: 42, CDR-H3 comprises the amino acid sequence of SEQ ID NO: 43, CDR-L1 comprises the amino acid sequence of SEQ ID NO: 45, CDR-L2 comprises the amino acid sequence of SEQ ID NO: 46, and CDR-L3 comprises the amino acid sequence of SEQ ID NO: 47;
(g) CDR-H1 comprises the amino acid sequence of SEQ ID NO: 92, CDR-H2 comprises the amino acid sequence of SEQ ID NO: 109, CDR-H3 comprises the amino acid sequence of SEQ ID NO: 110, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 112;
(h) CDR-H1 comprises the amino acid sequence of SEQ ID NO: 92, CDR-H2 comprises the amino acid sequence of SEQ ID NO: 114, CDR-H3 comprises the amino acid sequence of SEQ ID NO: 94, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 116; or
(i) CDR-H1 comprises the amino acid sequence of SEQ ID NO: 123, CDR-H2 comprises the amino acid sequence of SEQ ID NO: 124, CDR-H3 comprises the amino acid sequence of SEQ ID NO: 94, CDR-L1 comprises the amino acid sequence of SEQ ID NO: 53, CDR-L2 comprises the amino acid sequence of SEQ ID NO: 54, and CDR-L3 comprises the amino acid sequence of SEQ ID NO: 55.

32. The method of claim 31, wherein the antibody comprises a heavy chain variable domain (VH) amino acid sequence having at least 90% identity to a sequence selected from SEQ ID NO: 4, 12, 20, 28, 36, 44, 111, 115, and 125; and/or a light chain variable domain (VL) amino acid sequence having at least 90% identity to a sequence selected from SEQ ID NO: 8, 16, 24, 32, 40, 48, 56, 113, and 117; optionally, wherein:
(a) the antibody comprises a VH amino acid sequence having at least 90% identity to SEQ ID NO: 4; and a VL amino acid sequence having at least 90% identity to SEQ ID NO: 8;
(b) the antibody comprises a VH amino acid sequence having at least 90% identity to SEQ ID NO: 12; and a VL amino acid sequence having at least 90% identity to SEQ ID NO: 16;
(c) the antibody comprises a VH amino acid sequence having at least 90% identity to SEQ ID NO: 20; and a VL amino acid sequence having at least 90% identity to SEQ ID NO: 24;
(d) the antibody comprises a VH amino acid sequence having at least 90% identity to SEQ ID NO: 28; and a VL amino acid sequence having at least 90% identity to SEQ ID NO: 32;
(e) the antibody comprises a VH amino acid sequence having at least 90% identity to SEQ ID NO: 36; and a VL amino acid sequence having at least 90% identity to SEQ ID NO: 40;
(f) the antibody comprises a VH amino acid sequence having at least 90% identity to SEQ ID NO: 44; and a VL amino acid sequence having at least 90% identity to SEQ ID NO: 48;
(g) the antibody comprises a VH amino acid sequence having at least 90% identity to SEQ ID NO: 111; and a VL amino acid sequence having at least 90% identity to SEQ ID NO: 113;
(h) the antibody comprises a VH amino acid sequence having at least 90% identity to SEQ ID NO: 115; and a VL amino acid sequence having at least 90% identity to SEQ ID NO: 117; or
(i) the antibody comprises a VH amino acid sequence having at least 90% identity to SEQ ID NO: 125; and a VL amino acid sequence having at least 90% identity to SEQ ID NO: 56.

33. The method of claim 31, wherein the antibody comprises a HC-IL-10 fusion amino acid sequence having at least 90% identity to a sequence selected from SEQ ID NO: 80, 81, 82, 134, 136, and 140, and a light chain (LC) amino acid sequence having at least 90% identity to a sequence selected from SEQ ID NO: 135, 137, 141, 143, 145, and 147; optionally, wherein
(a) the HC-IL-10 fusion amino acid sequence of SEQ ID NO: 80, and the LC amino acid sequence of SEQ ID NO: 143;
(b) the HC-IL-10 fusion amino acid sequence of SEQ ID NO: 81, and the LC amino acid sequence of SEQ ID NO: 145;
(c) the HC-IL-10 fusion amino acid sequence of SEQ ID NO: 82, and the LC amino acid sequence of SEQ ID NO: 147;
(d) the HC-IL-10 fusion amino acid sequence of SEQ ID NO: 134, and the LC amino acid sequence of SEQ ID NO: 135;
(e) the HC-IL-10 fusion amino acid sequence of SEQ ID NO: 136, and the LC amino acid sequence of SEQ ID NO: 137; or
(f) the HC-IL-10 fusion amino acid sequence of SEQ ID NO: 140, and the LC amino acid sequence of SEQ ID NO: 141.

34. The method of claim 1, wherein:
(a) the protein increases MC/9 cell proliferation by at least 25%, at least 50%, at least 100%, at least 150%, at least 200% or more;
(b) the protein increases IFNγ and granzyme B production from activated CD8 T cells by at least 25%, at least 50%, at least 100%, or more; and/or
(c) the antibody decreases tumor volume in a syngeneic mouse tumor model measured at 28 days by at least 25%, at least 50%, at least 75%, or more, wherein the mouse tumor model is selected from: CT26 colon cancer and EMT6 breast cancer.
